(19) United States
(12) Patent Application Publication
(10) Pub. No.: US 2026/0038118 A1
(43) Pub. Date: Feb. 5, 2026

(54) SYSTEMS AND METHODS FOR THE QUANTITATIVE ASSESSMENT OF AIRWAY MUCUS PLUG PATHOLOGY

(71) Applicant: The Regents of the University of California, Oakland, CA (US)

(72) Inventors: John Fahy, San Francisco, CA (US); Brett Elicker, Novato, CA (US); Travis Henry, Durham, NC (US); Brendan Huang, San Francisco, CA (US)

(21) Appl. No.: 19/298,813

(22) Filed: Aug. 13, 2025

Related U.S. Application Data
(63) Continuation of application No. PCT/US2024/021014, filed on Mar. 21, 2024.
(60) Provisional application No. 63/454,002, filed on Mar. 22, 2023.

(51) Int. Cl. G06T 7/00; A61B 5/00; A61B 5/08; G06T 7/11; G06T 7/155; G06T 7/62

(52) U.S. Cl. CPC G06T 7/0012; A61B 5/08; A61B 5/4848; A61B 5/7475; G06T 7/11; G06T 7/155; G06T 7/62; A61B 2560/0295; G06T 2207/10081; G06T 2207/20044; G06T 2207/20081; G06T 2207/20084; G06T 2207/30061

(57) ABSTRACT

Systems and methods for the improved quantitative assessment of airway mucus plug pathology are provided herein which provide a quantitative suite of mucus imaging metrics that objectively quantifies features of mucus plug pathology in the lungs. Systems and methods for generating a subject-level mucus plug map that illustrates the position of one or more whole mucus plugs, or the absence of mucus plugs in an airway tree are discussed. Systems and methods for the determination of a subject-level mucus plug phenotype are provided herein.

### CROSS REFERENCE TO RELATED APPLICATIONS

This application is a continuation, filed under 35 U.S.C. 120, of International Patent Application No. PCT/US2024/021014 filed on Mar. 21, 2024, which claims priority to and the benefit of U.S. Provisional Application No. 63/454,002 filed on Mar. 22, 2023, the entire contents of which are incorporated by reference herein and all purposes, including any drawings.

### STATEMENT REGARDING FEDERALLY SPONSORED RESEARCH OR DEVELOPMENT

This invention was made with government support under P01 HL107202, R01 HL080414, and P01 HL128191 awarded by the National Institutes of Health. The government has certain rights in the invention.

### TECHNICAL FIELD

The subject matter described herein relates to systems and methods for the quantitative assessment of airway mucus plug pathology.

### BACKGROUND

Mucus, an extracellular gel composed of water and mucins (heavily glycosylated proteins) serves as a protectant barrier for the lungs. However, the presence of excessive mucus or impaired clearance of mucus is associated with common airway diseases. Indeed, abnormal mucus pathology is associated with diseases such as asthma, chronic obstructive pulmonary disease (COPD), acute bronchitis, chronic bronchitis, bronchiectasis, allergic bronchopulmonary aspergillosis, primary ciliary dyskinesia, and cystic fibrosis.

Mucus plugs, or mucus that occludes any of the airways of the lungs, can reduce airflow, and thus provides an attractive target for novel therapeutic interventions. However, identifying mucus plugs within a patient remains an intensive process that requires highly trained radiologists to review hundreds of images manually.

Additionally, even if conventional systems were able to identify mucus plugs, they provide little to no characterization of the underlying mucus plug and limited information on the mucus plugs themselves. Accordingly, there is limited information provided by conventional systems that may lead to treatment selection or provide information regarding how effective a treatment for a common airway disease is.

### SUMMARY

Systems and methods for the quantitative assessment of airway mucus pathology are provided herein. The quantitative assessment of airway mucus pathology may provide better treatment options to patients having common airway diseases.

In general, in an aspect, a plurality of images of one or both lungs of a subject from a computed tomography scan may be received. One or more mucus plug portions may be identified within the received plurality of images. A whole mucus plug associated with each of the identified mucus plug portions may be determined by applying a continuity algorithm to the received plurality of images. At least one whole mucus plug metric may be generated for each of the determined whole mucus plugs, where the whole mucus plug metric includes at least one of a length, a diameter, a volume, or a surface area. At least one whole mucus plug phenotype may be determined for each of the determined whole mucus plugs. Subject-level whole mucus plugs metrics including at least one of an average length, an average diameter, an average volume, or an average surface area may be generated for the determined whole mucus plugs. A subject-level mucus characterization data set including at least one of a whole mucus plug score, a total mucus plug volume, and a mucus plug slice score may be determined. A subject-level quantitative assessment of airway mucus plug pathology may be generated based on the subject-level mucus characterization data set. Subject-level mucus plug phenotype may be determined based on the plurality of generated whole mucus plug phenotypes.

Implementations may include one or a combination of two or more of the following features. Generating the at least one whole mucus plug phenotype may include applying a threshold value to at least one of a length, a diameter, a volume, or a surface area, or a combination thereof, in order to determine a categorization of each mucus plug. Generating a subject-level mucus characterization data set includes determining at least one of an average length, an average diameter, an average volume, or an average surface area for the determined whole mucus plugs. The efficacy of treatments based on the determined subject-level quantitative assessment of airway mucus path pathology can be predicted or monitored and the treatment may include at least one of a muco-active drug, an anti-inflammatory drug, interventional bronchoscopy, a cough assist device, or a vibrating vest. The efficacy of treatments based on the determined subject-level mucus plug phenotype can be predicted or monitored and the treatment may include at least one of a muco-active drug, an anti-inflammatory drug, interventional bronchoscopy, a cough assist device, or a vibrating vest. The whole mucus plug score may be indicative of the total number of whole mucus plugs across the plurality of images associated with the subject. The total mucus plug volume may include an aggregation of volume for each of the determined whole mucus plus for the subject. The mucus plug slice score includes the sum of the mucus plug annotations across the received plurality of images. Plug-level phenotypes may include a stubby mucus pattern where a stubby mucus pattern indicates at least one of a length less than a predetermined threshold or a ratio of length-diameter less than a predetermined threshold. Plug-level phenotypes may include a stringy mucus pattern, wherein a stringy mucus pattern indicates a length greater than a second predetermined threshold or a ratio of length-diameter greater than a second predetermined threshold. A subject-level phenotype may include one or more plugs classified within each phenotype. Generating an airway mucus plug map may include depicting a specific airway location for the determined whole mucus plugs, and determining the position of the one or more whole mucus plugs, or the absence of said one or more mucus plugs in relation to an airway tree. Generating the airway mucus map may also include segmenting the plurality of images to identify a plurality of pixels depicting the lung and a plurality of airways included in the lungs, determining for each whole mucus plug a distance between the whole mucus plug and an endpoint of each airway in the plurality of airways, assigning based at least on the distance between the whole mucus plug and the endpoint of each airway each whole mucus plug to an airway having a nearest airway termination point, and depicting the plurality of airways and the one or more mucus plugs assigned to the airway having the nearest airway termination point. Airway map generation may also include the steps of skeletonizing the plurality of airways to determine a best fit centerline for each airway of the plurality of airways, and determining based at least on the skeletonized plurality of airways the location of the endpoint of each airway. Generating the airway mucus plug map may include depicting a hierarchy of the plurality of airways. The airway mucus plug map may also include depicting a lobar location and generation of each airway of the plurality of airways. Identifying one or more mucus plug portions within the received plurality of images may include receiving an annotation identifying at least the portion of the one or more mucus plug portions. The annotation may be received from a client device and include one or more user inputs placing a mark around at least the portion of the mucus plug portions in the one or more received images, and determining based at least on the one or more user inputs the annotation identifying one or more mucus plug portions in the one or more images.

Identifying one or more mucus plug portions within the received plurality of images may include applying a trained machine learning algorithm on the received plurality of images. The machine learning algorithm may include a convolutional neural network or a transformer model. Determining a whole mucus plug associated with each of the identified mucus plug portions may include applying a set of algorithms to identify voxel-members of the whole mucus plug that form a region of interest, wherein the set of algorithms comprises one or more of a trained clustering algorithm, or sequential slicing annotation algorithms, and selecting one or more contiguous regions of interest. Determining a whole mucus plug associated with each of the identified mucus plug portions may include determining a region of interest for the identified mucus plug portion for each of image among the plurality of images, wherein the region of interest separates a foreground corresponding to the mucus plug portion and a background corresponding to a parenchyma of the lung based on cluster analysis. The plurality of images may include any cross-sectional imaging modality computed tomography (CT) scan of the lung. Each image of the plurality of images may include an axial, coronal, or sagittal slice of the computed tomography (CT) scan.

In general, in an aspect, a method may include receiving a plurality of images of one or both lungs of a subject from a computed tomography scan, identifying one or more mucus plug portions within the received plurality of images, determining a whole mucus plug associated with each of the identified mucus plug portions by applying a continuity algorithm to the received plurality of images, determining a specific airway location for the determined whole mucus plug, and generating an airway mucus plug map depicting a specific airway location for the determined whole mucus plugs.

Implementations may include one or a combination of two or more of the following features. Generating the airway mucus map may include determining the position of the one or more whole mucus plugs, or the absence of said one or more whole mucus plugs in relation to an airway tree. Generating the airway mucus map may further include segmenting the plurality of images to identify a plurality of pixels depicting the lung and a plurality of airways included in the lungs, determining, for each whole mucus plug, a distance between the whole mucus plug and an endpoint of each airway in the plurality of airways, assigning, based at least on the distance between the whole mucus plug and the endpoint of each airway, each whole mucus plug to an airway having a nearest airway termination point, and depicting the plurality of airways and the one or more mucus plugs assigned to the airway having the nearest airway termination point. The method may also include skeletonizing the plurality of airways to determine a best fit centerline for each airway of the plurality of airways, and determining based at least on the skeletonized plurality of airways the location of the endpoint of each airway. Generating the airway mucus plug map may include depicting a hierarchy of the plurality of airways, including for example, depicting a lobar location and generation of each airway of the plurality of airways.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that can include one or more processors and one or more memories coupled to the one or more processors. A memory, which can include a non-transitory computer-readable or machine-readable storage medium, can include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to web application user interfaces, it should be readily understood that such features are not intended to be limiting. The claims that follow the disclosure are intended to define the scope of the protected subject matter.

### BRIEF DESCRIPTION OF THE DRAWINGS

FIG. 1 illustrates a set of lungs for a subject, in accordance with some embodiments of the present disclosure.

FIG. 2 illustrates a process for quantitative analysis of mucus plugs, in accordance with some embodiments of the present disclosure.

FIG. 3A illustrates a process for quantitative analysis of mucus plugs, in accordance with some embodiments of the present disclosure.

FIG. 3B illustrates a process for generating a subject-level mucus plug map of whole mucus plugs, in accordance with some embodiments of the present disclosure.

FIG. 4 provides an illustration of an annotated CT scan image in accordance with some embodiments of the present disclosure.

FIG. 5 provides a visualization of a whole mucus plug in accordance with some embodiments of the present disclosure.

FIG. 6 provides a visualization of various phenotypes of mucus plugs in accordance with some embodiments of the present disclosure.

FIG. 7 illustrates correspondence between mucus plug volume and airflow obstruction in accordance with some embodiments of the present disclosure.

FIG. 8 illustrates a mapping of mucus plugs throughout a subject's lungs in accordance with some embodiments of the present disclosure.

FIG. 9 illustrates a workflow for annotation and segmentation of mucus plugs in accordance with some embodiments of the present disclosure.

FIG. 10 illustrates an image processing pipeline in accordance with some embodiments of the present disclosure.

FIGS. 11A-11C illustrate the processes by which quantitative assessment of airway mucus plug pathology metrics may be generated in accordance with some embodiments of the present disclosure.

FIGS. 12A-12D provide an overview of an image processing pipeline for mucus plug localization and mucus plug airway map generation in accordance with some embodiments of the present disclosure.

FIGS. 13A-13F illustrate the display of mucus plug characterization data in accordance with some embodiments of the present disclosure.

FIG. 14 illustrates the automated detection of mucus plug portions in accordance with some embodiments of the present disclosure.

FIG. 15 provides an illustration of results from the automated detection of mucus plug portions in accordance with some embodiments of the present disclosure.

FIG. 16 illustrates a computer system for use in accordance with some embodiments of the present disclosure.

FIG. 17 provides an example system diagram for a quantitative assessment of airway mucus pathology in accordance with some embodiments of the present disclosure.

FIGS. 18A-18E provide experimental data in accordance with some embodiments of the present disclosure.

FIGS. 19A-19C provide experimental data in accordance with some embodiments of the present disclosure.

FIGS. 20A-20F provide experimental data in accordance with some embodiments of the present disclosure.

FIGS. 21A-21B provide experimental data in accordance with some embodiments of the present disclosure.

FIGS. 22A-22C provide experimental data in accordance with some embodiments of the present disclosure.

FIGS. 23A-23J provide experimental data in accordance with some embodiments of the present disclosure.

FIGS. 24A-24B provide experimental data in accordance with some embodiments of the present disclosure.

FIGS. 25A-25K provide experimental data in accordance with some embodiments of the present disclosure.

FIGS. 26A-26B provide experimental data in accordance with some embodiments of the present disclosure.

### DETAILED DESCRIPTION

The presence of excessive mucus, or impaired clearance of mucus, is a pathologic feature of multiple airway diseases. Excess mucus has several consequences for lung health including the impairment of airflow and increased susceptibility to infection. Abnormal mucus pathology is associated with common respiratory diseases such as asthma, chronic obstructive pulmonary disease (COPD), acute bronchitis, chronic bronchitis, bronchiectasis, allergic bronchopulmonary aspergillosis, primary ciliary dyskinesia, and cystic fibrosis. Recent advancements in imaging technology have demonstrated that there are mucus plug-high sub-phenotypes of asthma and COPD that are visible via computed tomography (CT) scans of the lungs.

Although mucus plugs, which are collections of mucus that occlude any of the airways and reduce airflow, provide an attractive target for novel therapeutic interventions, conventional systems have been unable to identify mucus plugs efficiently and at large scales. For example, conventional methods of identifying mucus plugs within a patient remains an intensive process that requires highly trained radiologists to review hundreds of images manually. Prior approaches have been limited by the challenges presented by the bronchopulmonary anatomy, where the vast segmentation within the anatomy prevents a challenge to automated techniques. Additionally, the inability to automate characterization of mucus plugs across lung scans prohibits the generation of population-level statistics and data that may be required for the development and/or improvement of treatments.

Further, conventional systems have been unable to characterize mucus plugs in a manner that allows for the development of a treatment plan or for monitoring whether a treatment is working. Some have created a mucus segment score which looks at mucus plug burden based on the presence of at least one mucus-occluded airway in a bronchopulmonary segment. However, this metric provides limited information as to the overall mucus plug burden on a patient, mucus location, or mucus plug size. For example, without information regarding the positioning of mucus plugs or how expansive the size of the mucus plugs are, it is difficult to tailor inhaled aerosol treatments so that such aerosol treatments deposit in the correct airway locations, or determine the required dosage for muco-active drugs. Additionally, the decision whether to intervene via a directed mechanical procedure such as bronchoscopy as opposed to use of a non-invasive mechanical therapy such as vest therapy can also be guided by data regarding the positioning and size of mucus plugs.

Systems and methods for the improved quantitative assessment of airway mucus pathology are provided herein. In some embodiments, a comprehensive quantitative assessment of airway mucus plug pathology (“qAAMP”) is provided that produces a suite of mucus imaging metrics that objectively quantifies multiple features of mucus plug pathology in the lungs.

Systems and methods for the generation of a subject-level mucus plug map that illustrates the position of one or more whole mucus plugs, or the absence of mucus plugs in an airway tree are provided herein.

Additionally, systems and methods for the determination of a subject-level mucus plug phenotype are provided herein.

In some embodiments a plurality of images of one or both lungs of a subject from a computed tomography (CT) scan may be received. One or more mucus plug portions may be identified from within the received plurality of images. A continuity algorithm may be applied to the received plurality of images to determine a whole mucus plug associated with each identified mucus plug portion. At least one whole mucus plug metric may be generated for each of the determined whole mucus plugs. For example, whole mucus plug metrics may include at least one of a length, a diameter, a volume or a surface area. A whole mucus plug phenotype may be generated for each of the determined whole mucus plugs. In some embodiments, the whole mucus plug phenotype may be generated by applying a threshold measure of at least one of a length, a diameter, a volume, a surface area, or a combination thereof, in order to categorize each mucus plug. Subject-level whole mucus plug metrics may be generated based on at least one of an average length, average diameter, average volume, or average surface area for the determined whole mucus plugs. A subject-level mucus characterization data set may be generated and include at least one of a whole mucus plug score, a total mucus plug volume, and a mucus plug slice score. A subject-level mucus plug map may illustrate the position of one or more whole mucus plugs or the absence of mucus plugs in relation to an airway tree. A subject-level quantitative assessment of airway mucus plug pathology may be determined based on the subject-level mucus characterization data set. Additionally, a subject-level mucus plug phenotype may be determined based on the plurality of generated whole mucus plug phenotypes.

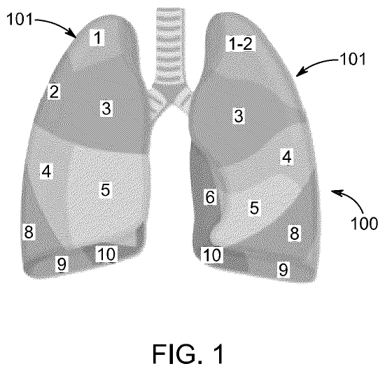

FIG. 1 provides a schematic representation 100 of a subject's lungs. Left and right lungs 101 are depicted. As shown, each lung 101 may have a plurality of segments. Together the lungs have twenty (20) broncho-pulmonary segments. The anatomy illustrated in the schematic representation 100 is challenging for computerized models to navigate.

Prior work describes a mucus segment score that rates mucus plug burden based solely on the presence or absence of any mucus plug within a broncho-pulmonary segment. Mucus segment scores range from 0 to 20 and correlate to the 20 broncho-pulmonary segments illustrated in FIG. 1. Limitations of the mucus segment score include that it is semi-quantitative, provides a limited amount of information about mucus plug burden and location, and provides no information about the size dimensions of each mucus plug. In addition, the mucus segment score is difficult to automate, which precludes scaling it to measures of mucus plugs in large numbers of multidetector row computed tomography (MDCT) scans of the lungs.

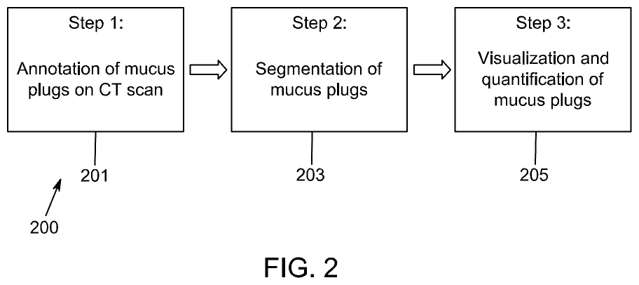

FIG. 2 provides an illustration for a process 200 for quantitative analysis of mucus plugs. In a first step 201, images of lungs with annotations indicating the presence of mucus plug portions may be obtained. In some embodiments, the images may form a plurality of images received from a computed tomography (CT) scan. In some embodiments, the plurality of images include images from any cross-sectional imaging modality. In some embodiments, each image of the plurality of images may include an axial, coronal, or sagittal slice of the CT scan.

In a second step 203, the identified mucus plug portions may be segmented such that a corresponding whole mucus plug may be associated with the identified mucus plug portion.

In a third step 205, mucus plugs may be visualized and quantified. For example, mucus plugs may be visualized in a three-dimensional rendering. In another example, mucus plugs may be visualized in a subject-level mucus plug map that visually represents the position of determined whole mucus plugs in relation to an airway tree.

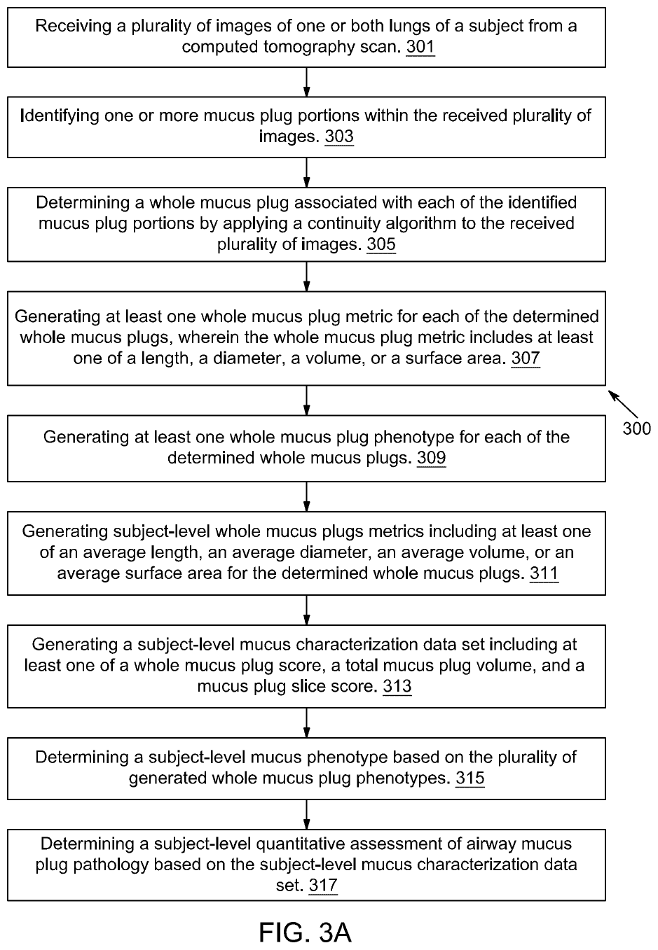

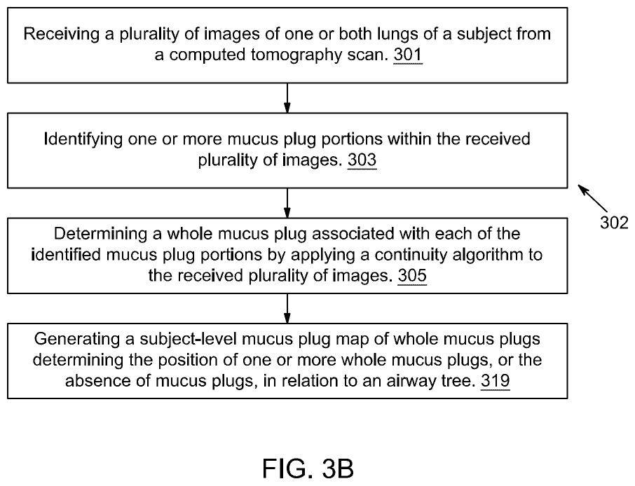

FIG. 3A provides an illustration for a process 300 for quantitative analysis of mucus plugs. In a first step 301, a plurality of images of one or both lungs of a subject from a computed tomography scan are received. In some embodiments, the images may be from a scan of a single lung from a subject, or from both lungs of from a subject. In some embodiments, the scans may include multi-detector row computed tomography (MDCT) scans of the lungs. In some embodiments, the images may be from a computed tomography (CT) scan. In some embodiments, the plurality of images include images from any cross-sectional imaging modality. In some embodiments, each image of the plurality of images may include an axial, coronal, or sagittal slice of the CT scan. Although images from a CT scan are discussed herein, it is envisioned that in some embodiments alternative imaging modalities such as x-rays, magnetic resonance imaging, and the like may be used to obtain the images. Depending on the size of the patient and the specifics of the CT lung scan protocol, imaging technology may yield 400-700 or more axial lung slices for a subject or patient.

In some embodiments, the received annotations may be provided by a radiologist or other medical professional via a client device configured to display images from a CT scan and receive markings of mucus plug portions. In such an embodiment, a radiologist or other user may place a mark around at least a portion of a mucus plug present in the provided image. The annotation may be logged and provided to an image processing pipeline.

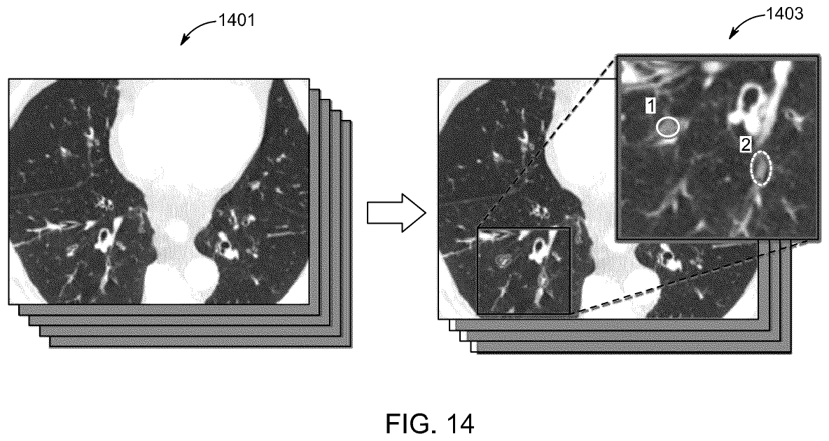

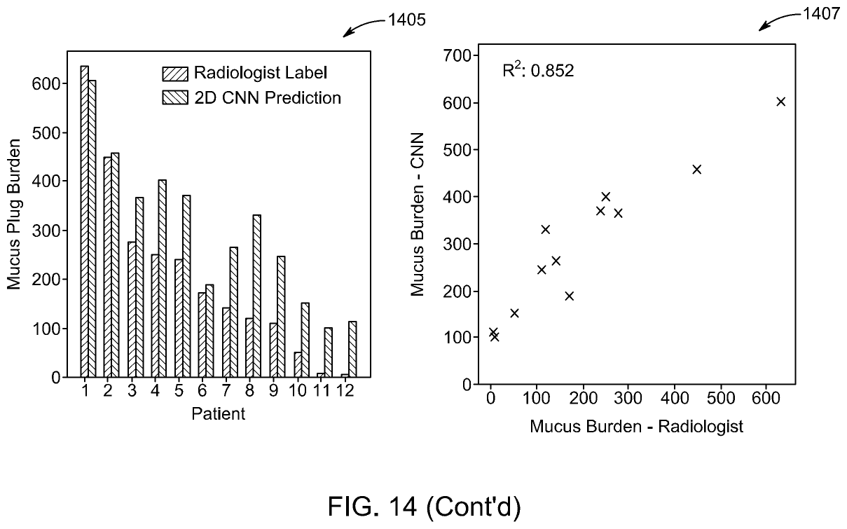

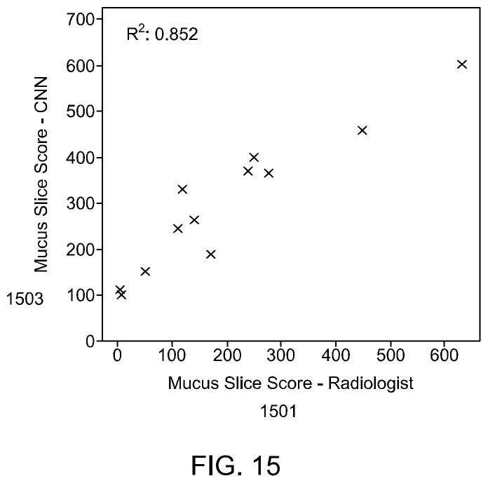

In some embodiments, the received annotations may be automatically generated by an image processing system. For example, the image processing pipeline may utilize a trained machine learning algorithm as described in FIGS. 14 and 15 (below) to automatically identify mucus plug portions within a provided image.

In a second step 303 of the process 300, one or more mucus plug portions may be identified from within the received plurality of images. In some embodiments, an image processing pipeline may include an algorithm configured to identify mucus plug portions in images. The algorithm may be configured to locate the annotations present in the image.

In a third step 305 of the process 300, the corresponding whole mucus plug for each of the identified mucus plug portions may be determined by applying a continuity algorithm to the received plurality of images. In some embodiments, a set of algorithms may be applied to identify the voxel-members of the whole mucus plug located within a slice. The voxel-members may form a region of interest. The set of algorithms may include image processing algorithms that are configured to identify and separate a foreground that corresponds to the mucus plug portion from a background that may correspond to the parenchyma of the lung. In some embodiments, the image processing algorithms may include a trained cluster analysis algorithm. Further, a sequential slicing annotation algorithm may be used to select continuous regions of interest across the various images. Because the provided images show a slice of the lung, a whole mucus plug may span across a plurality of images (e.g., often 10 to 50 images), and so a whole mucus plug may be associated across a high number of images. These algorithms allow the ability to automatically generate mucus plug quantification data on large numbers of scans that are slow or impractical to implement manually. Application of automated methodology allows mucus plug quantification metrics to be generated quickly and at scale to enable research or clinical applications that are not otherwise possible.

In a fourth step 307, at least one whole mucus plug metric may be generated for each of the determined whole mucus plugs. In some embodiments, the whole mucus plug metric may include any metric capable of characterizing the whole mucus plug. For example, this may include a length, a diameter, a volume, or a surface area for the whole mucus plug.

In a fifth step 309, a whole mucus plug phenotype can be generated for a determined whole mucus plug. A phenotype of the whole mucus plug may describe the shape and size of mucus plugs formed within an airway. In some embodiments, each mucus plug can be categorized by a phenotype such as a sparse mucus pattern, a stubby mucus pattern, a stringy mucus pattern, or a branching mucus pattern. In some embodiments, the categorization into the various phenotypes may be based on the count, length, diameter, volume, surface area, or a ratio or threshold thereof. In some embodiments, a sparse mucus pattern may be associated with a mucus plug count of less than three. In some embodiments, the phenotype may be based on the ratio between the length and diameter of the whole mucus plug. For example, a stubby mucus pattern may be associated with length less than approximately 5-15 mm or a ratio of length to diameter less than approximately 2:4 mm. A stringy mucus pattern may be associated with length greater than approximately 5-15 mm or a ratio of length to diameter of greater than approximately 2:4 mm. A branching mucus pattern may be associated with a whole mucus plug having a common section from which two separate sections emanate. The shape and size of the mucus plug can be quantified by extracting voxels for each mucus plug. In some embodiments, mucus plugs can be categorized into two phenotypes: short plugs that are 12 mm or less in length, which can be denoted as “stubby,” and long plugs that are more than 12 mm in length, denoted as “stringy.”

Various subject-level (i.e., across the subject) quantitative metrics may be generated based on the determination of the phenotypes and metrics for each of the whole mucus plugs present in a subject's lungs.

For example, in a sixth step 311, subject-level whole mucus plugs metrics may be determined. Subject-level whole mucus plug metrics may include, but are not limited to, the average length of mucus plugs in a subject, the average diameter of mucus plugs in a subject, the average volume of mucus plugs in a subject, or the average surface area for mucus plugs in a subject.

In another example, in a seventh step 313, subject-level mucus characterization data set may be generated. In some embodiments the subject-level mucus characterization data set may include at least one of a whole mucus plug score, a total mucus plug volume, and a mucus plug slice score. The whole mucus plug score may be indicative of the total number of whole mucus plugs across the plurality of images associated with the subject. The total mucus plug volume may be a summation or aggregation of the volume of whole mucus plugs for the subject. The mucus plug slice score may be a summation or aggregation of the whole mucus plug slice score across all the identified whole mucus plugs.

In an eighth step 315, subject-level quantitative assessment of airway mucus plug pathology may be determined based on the subject-level mucus characterization data set. This may include the average length, the average diameter, the average volume, or the average surface area for the determined whole mucus plugs for the subject. The subject-level quantitative assessment of the airway mucus plug pathology may provide a numeric indicator of the mucus plug burden for a subject. Examples of mucus plug burden indicators include: mucus plug slice score, total mucus plug volume, total mucus plug number, average mucus plug length, and average mucus plug volume. Such indicators may be used in research as predictive and monitoring biomarkers to test the efficacy of established or novel muco-active drugs, mucus clearance treatments (i.e. bronchoscopic removal, or vest) or anti-inflammatory treatments. They may also be used in clinical practice to guide treatment decisions for individual patients with mucus-associated lung disease.

In some embodiments, the quantitative assessment of airway mucus plug pathology (alternatively referred to as “qAAMP”) may include measures of total mucus plug burden in the lungs. This may include a mucus plug slice score that is generated based on the number of mucus occluded airways in each axial slice of a lung scan. In some embodiments, there may be approximately 400-700 axial slices per MCDT scan. The total mucus plug volume may be generated based on the sum of the volumes of all mucus plugs in the lungs for a subject. The total mucus plug number may be based on the total number of whole mucus plugs identified in the subject's lungs.

In a ninth step 317, a subject-level mucus plug phenotype may be generated based on the plurality of generated whole mucus plug phenotypes. For example, if the majority of whole mucus plug phenotypes are of a particular phenotype, then the subject-level mucus plug phenotype may be set to that particular phenotype. Some subjects may have a majority of whole-mucus plug phenotypes of one particular phenotype while having whole mucus plugs of different phenotypes (i.e., majority stringy mucus plugs corresponding to a stringy patient-phenotype but contains some stubby mucus plugs). Mucus plug phenotypes may be used to generate, validate, or modify treatment plans for a patient including the type of treatment and the time course for the treatment.

In some embodiments, the process 300 may include a step to determine a subject-level mucus plug map of whole mucus plugs, analogous to the process described in FIG. 3B.

FIG. 3B provides an illustration for a process 302 for generating a subject-level mucus plug map of whole mucus plugs. As illustrated in FIG. 3B, in some embodiments, a process 302 may include the steps of receiving a plurality of images of one or both lungs of a subject from a computed tomography scan 301, identifying one or more mucus plug portions within the received plurality of images 303, determining a whole mucus plug associated with each of the identified mucus plug portions by applying a continuity algorithm to the received plurality of images 305, and generating a subject-level mucus plug map of whole mucus plugs 319. In some embodiments, steps 301, 303, 305 may be analogous to that described with respect to FIG. 3A.

The subject-level map may be displayed to clinicians on a user interface display, created into medical reports, and the like. The subject-level map may be generated by determining the position of one or more whole mucus plugs, or the absence of mucus plugs, in relation to the airway tree of the subject. The subject-level map may also provide a visual representation of the airway location of each mucus plug in the lungs for airway generations 0 through 12.

Developing a subject-level mucus plug map of whole mucus plugs located within the lungs of a subject may include the steps of segmenting the plurality of images to identify a plurality of pixels depicting the lung and a plurality of airways included in the lungs, determining a distance between each identified whole mucus plug and an endpoint of each airway in the plurality of airways, assigning each whole mucus plug to an airway based at least on the distance between the whole mucus plug and the endpoint of each airway, and depicting the plurality of airways and the one or more mucus plugs assigned to the airway having the nearest airway termination point.

Lung segmentation algorithms may include segmentation of the lung parenchyma using intensity thresholding and region growing or deep learning, the identification of fissures based on analysis of the Hessian matrix, and the segmentation of each lung lobe based on fissure identification. Airway segmentation algorithms may include the application of intensity thresholding and region growing algorithms followed by explosion region control or deep learning.

Identifying the plurality of airways included in the images of the lungs may also include skeletonizing the plurality of airways to determine a best fit centerline for each airway of the plurality of airways (using previously established methods), and determining the location of the endpoint of each airway based on the skeletonization. Skeletonizing an airway may refer to the process of reducing a three-dimensional cylindrical volume to a center pathline in three dimensions.

A visual representation of the plurality of airways may indicate the hierarchy of airways within the lung. Further, the visual representation may also depict the lobar location and generation of each airway. The representations of the lung may be overlaid with positional information regarding the presence of the whole mucus plugs within the lung. Examples of visual representations are provided below in FIGS. 14 and 15. In some embodiments, a visual representation of the plurality of airways may be overlaid with metric information regarding the whole mucus plugs present within the lung. For example, color coding, shading, tables, or other infographics can be used to indicate the size, shape, or phenotype of the whole mucus plugs along with their position along the airways.

In some embodiments, a process may include generating any one or a combination of subject-level mucus characterization data set, subject-level mucus phenotypes, subject-level quantitative assessments and an airway mucus map.

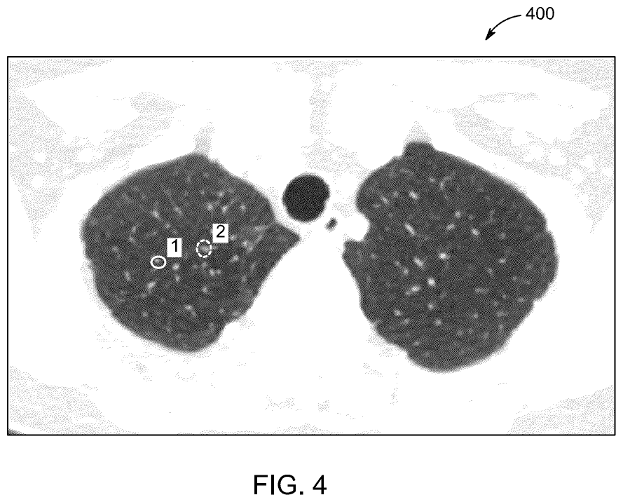

FIG. 4 provides an illustration of an annotated CT scan image in accordance with embodiments of the present disclosure. For example, the annotated CT scan image 400 may correspond to the annotations received in the first step 201 of the process for quantitative analysis of mucus plugs described in FIG. 2, or step 303 of the process illustrated in FIG. 3. As illustrated, annotations or marks may be placed over each mucus plug in an image. For example, elliptical marks may be placed over each mucus plug within an axial slice. The annotations may then be incorporated into an image-processing pipeline. In some embodiments the process of annotating or marking mucus plugs in an image can be automated using deep learning techniques. Further, plug segmentation can also be automated using deep learning and the volumetric segmentation data generated herein can be used to train analogous algorithms.

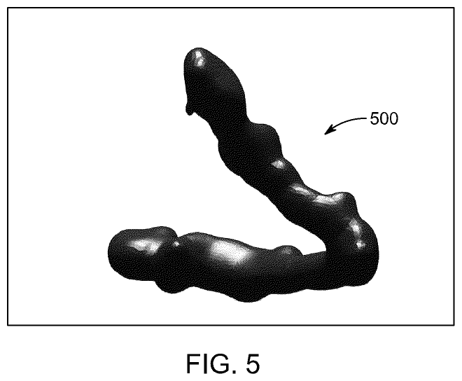

FIG. 5 provides a visualization of a whole mucus plug in accordance with embodiments of the present disclosure. A rendering of a whole mucus plug 500 is shown. While CT scan images may be annotated to indicate locations of mucus plug portions, in some embodiments, a whole mucus plug corresponding to the mucus plug portions present across various CT scan images may be generated.

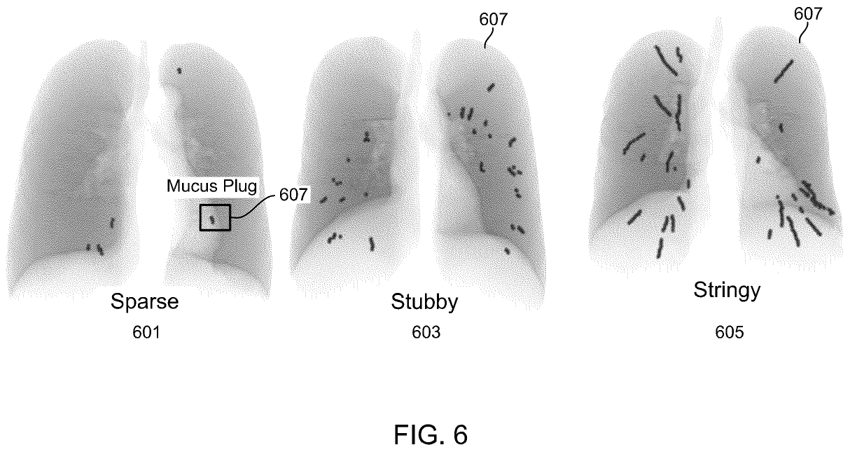

FIG. 6 provides a visualization of various phenotypes of mucus plugs. As illustrated, mucus plugs 607 may be dispersed throughout a subject's lungs. The mucus plugs 607 may have a sparse mucus pattern 601, a stubby mucus pattern 603, or a stringy mucus pattern 605.

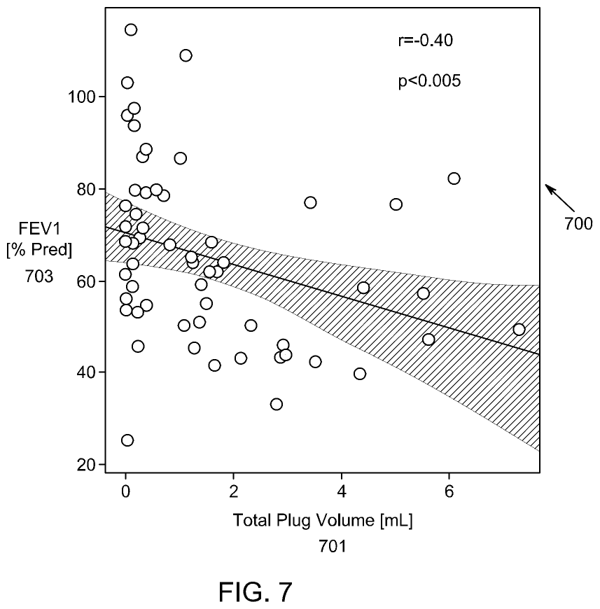

FIG. 7 illustrates correspondence between mucus plug volume and airflow obstruction. For example, as illustrated in chart 700, forced expiratory volume in 1 second (FEV1) 703 is correlated with total plug volume (mL) 701. The forced expiratory volume may be based on the maximum amount of air that a subject is able to forcibly expel during a time period (e.g., 1 second) following maximal inhalation. The total plug volume may be measured in mL or any other suitable metrics. Accordingly, increased mucus plug volume is a predictor of worse airflow obstruction.

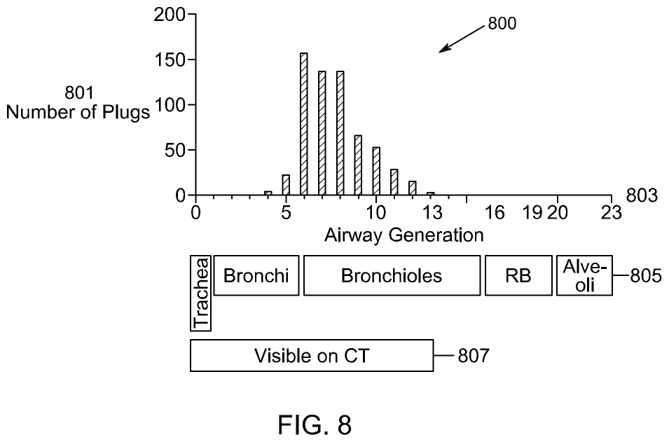

FIG. 8 illustrates a mapping of mucus plugs throughout a subject's lungs 800. Patient data was used to chart the number of plugs 801 present in each airway generation 803. As shown, experimental data showed that mucus plugs occupy generations 6-9 of the airway tree. The corresponding airway features of each generation 805 are mapped across airway generations 803. Further, only a portion of the airway generations may be visible on a CT scan 807. It is envisioned that the described systems and methods can be combined with future visualization techniques to construct further characterizations of mucus plugs. For example, three-dimensional geometry of airway mucus plugs can be reconstructed from sequences from two-dimensional CT lung images. Additionally, the location of mucus plugs in the airway tree can be determined using airway segmentation.

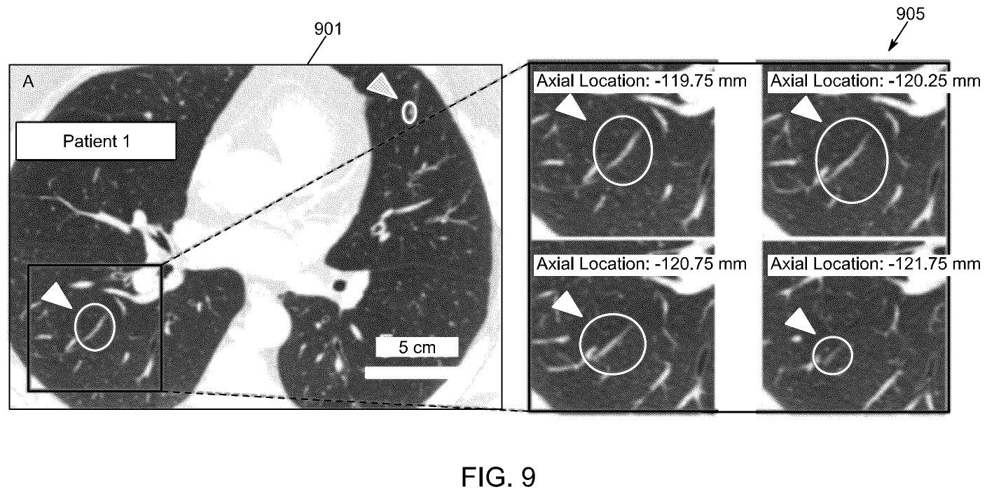

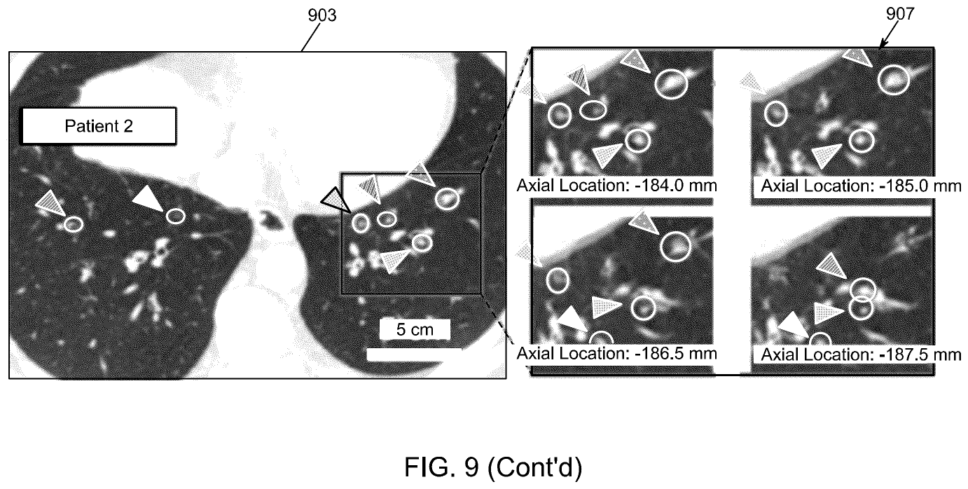

FIG. 9 illustrates a workflow for annotation and segmentation of mucus plugs for two patients. As shown an image or CT scan for a first patient 901 and an image or CT scan for a second patient 902 may be annotated. For example, elliptical marks may be present over each mucus plug portion identified within an axial slice 905, 907. The annotations may then be incorporated into an image processing pipeline. In some embodiments, the image may be obtained using multidetector row computed tomography (MDCT) scans of the lungs, in which a rotating X-ray tube and a row of detectors measure X-ray attenuations in the lungs. Multiple lung measurements from different angles may be processed using reconstruction algorithms to produce tomographic cross-sectional images or virtual “slices” of the lung. Depending on the size of the patient and the specifics of the CT lung scan protocol, this imaging technology may yield 400-700 axial lung slices for a patient.

In some embodiments, a radiologist may mark each mucus plug portion in each axial lung slice using annotation software and in this way a data file of slice-based mucus plug annotations is generated. For example, a thoracic radiologist may use a Digital Imaging and Communications (DICOM) viewer to place elliptical mark over each mucus plug within an axial slice of inspiratory MDCT images, generating a center coordinate, width, & height for a region of interest. The process may be repeated at each axial slice.

Alternatively, in some embodiments, a computer algorithm may be trained to process MDCT scans and generate regions of interest markings for each axial slice automatically as will be discussed with respect to FIGS. 14 and 15.

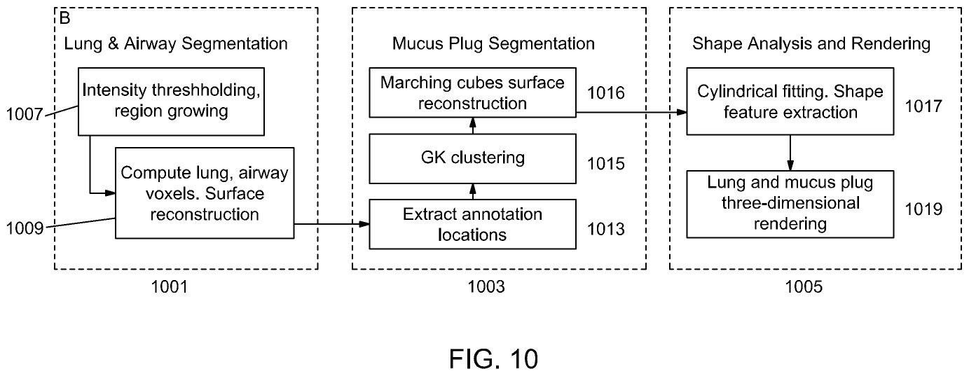

FIG. 10 illustrates an image processing pipeline. As illustrated, the image processing pipeline may include lung and airway segmentation 1001, mucus plug segmentation 1003, and shape analysis and rendering 1005. In some embodiments the annotations from either the radiologist or the computer algorithm may be processed by the image-processing pipeline.

Lung and airway segmentation 1001 may involve intensity thresholding and region growing 1007. For example, this may include performing intensity thresholding or binarizing the image based on either a predetermined or learned density cutoff in order to identify regions of interest. The regions of interest may be iteratively grown. For example, the region of interest may be expanded from a seed point in the lungs outward until the high intensity borders corresponding to the chest wall and abdominal viscera are reached. Intensity thresholding and region growing 1007 may include the selection of candidate lung and airway points based on low intensity or density and expanding outward. This many involve the use of deep learning-based segmentation algorithms, graph cut algorithms and diffeomorphic image registration from a lung atlas.

The lung and airway segmentation 1001 process may also include the extraction of regions of interest associated with each plug. A fuzzy-clustering technique may be used to separate high attenuating mucus as foreground and lung parenchyma as background. A surface mesh is generated and the mucus plug is segmented.

The lung and airway segmentation 1001 process may also include the computing of lung, and airway voxels as well as surface reconstruction 1009. This process may include identifying the specific voxels that are associated with lung tissue from an image.

The mucus plug segmentation 1003 process may also include the step of extracting annotation locations 1013. Extracting annotation locations may include registering annotation locations with DICOM images.

After the annotation locations are extracted, clustering 1015 may be performed. This may involve the use of a soft clustering technique (e.g., Gustafson-kessel (G-K) clustering) in order to separate foreground (i.e., mucus) from the background. Clustering 1013 may include the use of predetermined intensity cutoffs for binarization, Otsu's method, and/or hard clustering techniques.

Marching cubes surface reconstruction 1016 may be performed as a part of mucus plug segmentation 1003. In particular, surface elements may be extracted from voxels using Delaunay triangulation or other suitable methods.

The segmented mucus plugs may then be provided to a shape analysis and rendering process 1005. The shape analysis and rendering process 1005 may first perform cylindrical fitting and shape feature extraction 1017. In this process the shape of a mucus plug is estimated as a cylinder and fit to an effective radius and length. Cylindrical fitting and shape feature extraction may involve the use of principal component analysis on the shape to obtain major, minor, and least axis metrics. The largest and smallest distance of the set of surface points may be found.

In a last step, three-dimensional renderings for lung and mucus plugs are generated 1019. This may allow for the visualization of lung and mucus plugs in an optical projection of a three-dimensional surface. The process 1019 may involve showing segmented voxels in a z-stack of images.

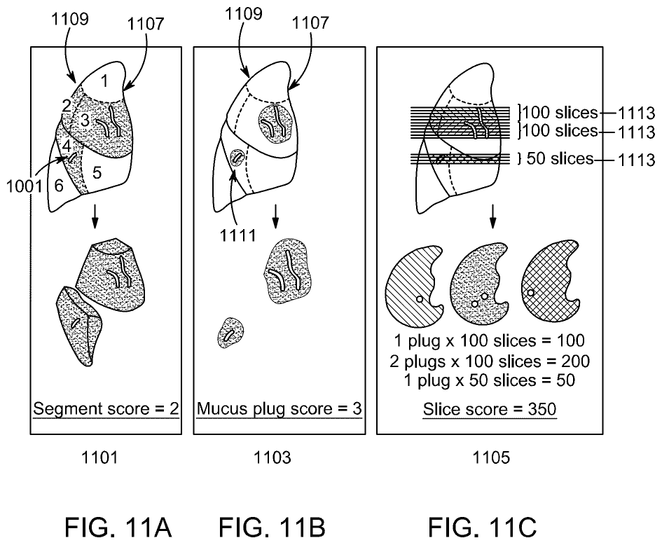

FIG. 11 illustrates the processes by which quantitative assessment of airway mucus plug pathology (alternatively referred to as “qAAMP”) metrics may be generated. For example, a segment score 1101, mucus plug score 1103 and slice score 1105 may be generated for each subject. As illustrated, a subject may have a lung 1107 with a plurality of mucus plugs 1111 dispersed within the lung 1107 within bronchopulmonary segments 1109. The segment score 1101 may correspond to the number of broncho-pulmonary segments 1109 that contain mucus plugs in the subject. The mucus plug score 1103 may correspond to the total number of mucus plugs 1111 across the subject. The slice score 105 may correspond to the number of slice images 1113 that include plug segments. Together, the segment score 1101, mucus plug score 1103, and slice score 1105 may provide an indication of the mucus plug burden placed on a subject. In some embodiments, the slice score 1105 may be generated automatically by a software program. For example, an annotation data file may be used to compute the mucus slice score by summing all mucus plug annotations in the patient's stack of axial lung slices. The mucus slice score provides a highly quantitative measure of mucus plug burden in the lung. For example, in some embodiments, a clinician marks CT scans with elliptical markings on airways occluded by mucus. Subsequently a clustering algorithm can be applied to the elliptical annotations. A point can be assigned for each elliptical annotation, and a patient-specific “mucus slice score” can be generated from the sum of the points. The mucus slice score can provide a quantitative indication of mucus burden. Further the total number of discrete mucus plugs per patient can be denoted as the mucus plug score.

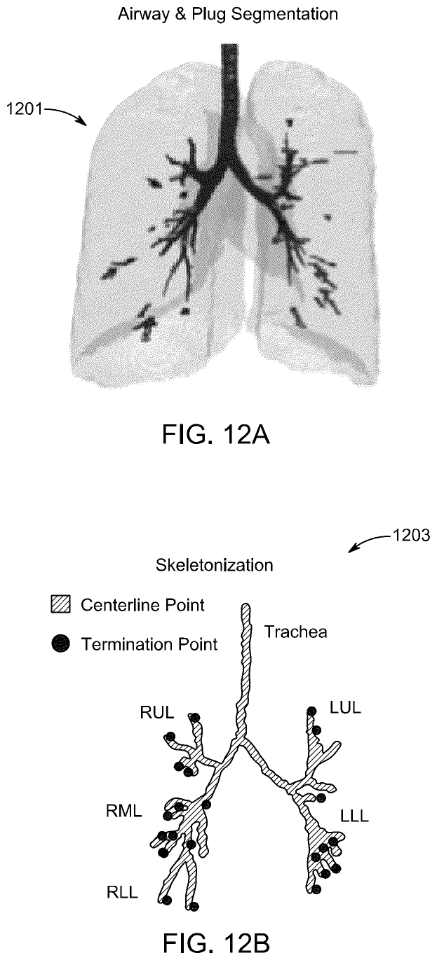

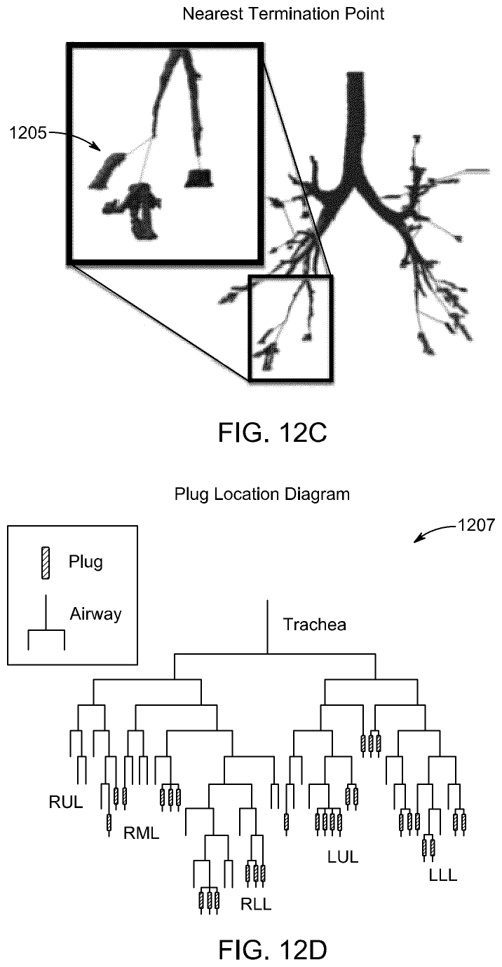

FIGS. 12A-12D provide an overview of an image processing pipeline for mucus plug localization and mucus plug airway map generation. In some embodiments, the mucus plug airway map may be patient specific, and present a representation of the subject's airway system. In a first step 1201 airways and lungs may be segmented by applying an intensity thresholding and region growing or deep learning algorithm to images of the lung derived from the a CT image. In some embodiments, lungs may be segmented by lobe. The segmented plugs may be co-registered with the lungs. In a second step 1203, airways may be skeletonized by determining a best fit centerline by using a distance transform technique. The ends of each airway may be labeled as a termination point or terminal airway. As shown, the lungs may be separated into a trachea, right upper lobe, left upper lobe, right middle lobe, left lower lobe, right lower lobe. In a third step 1205 the nearest termination point may be determined for each whole mucus plug by computing a distance between each termination point and every point at the surface of any given mucus plug. The distance metric may be a Euclidian distance or a distance that penalizes traversing lung high intensity pixels such as airway walls and lung parenchyma. In a fourth step 1207, a plug location diagram may be generated that indicates the position of each plug within the airway map. As illustrated, the airway mucus plug map may provide a representation of each generation of the airway, lobar locations, and each of the airways associated with a whole mucus plug.

In some embodiments the airway mucus plug map may provide positional data to a user for all of the locations of whole mucus plugs within the lungs. The airway mucus plug map may be used to better understand the pathology of airway mucus plug formation, and for the development of therapeutics for mucus-associated lung diseases such as asthma, chronic obstructive pulmonary disease (COPD), acute bronchitis, chronic bronchitis, bronchiectasis, allergic bronchopulmonary aspergillosis, primary ciliary dyskinesia, and cystic fibrosis. For example, the location of airway mucus plugs may guide responses to muco-active drugs.

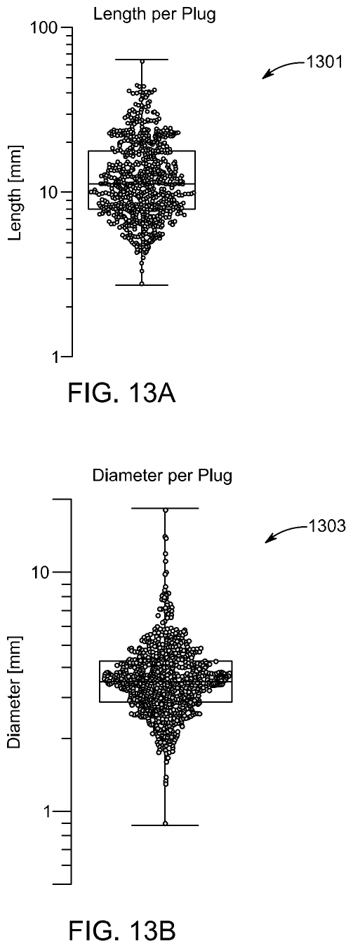

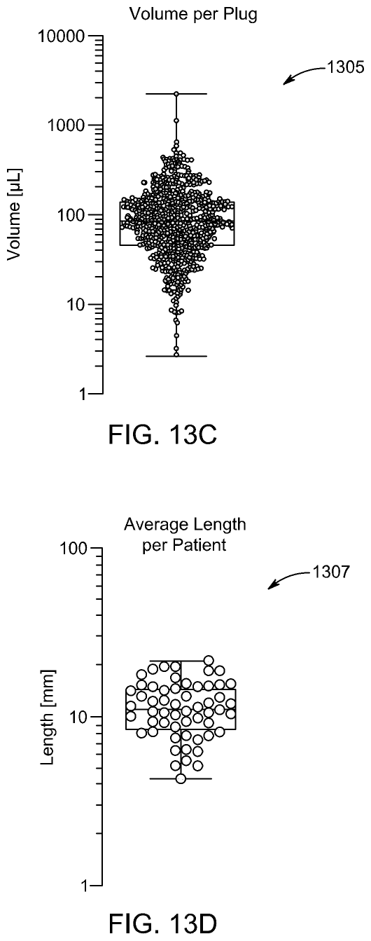

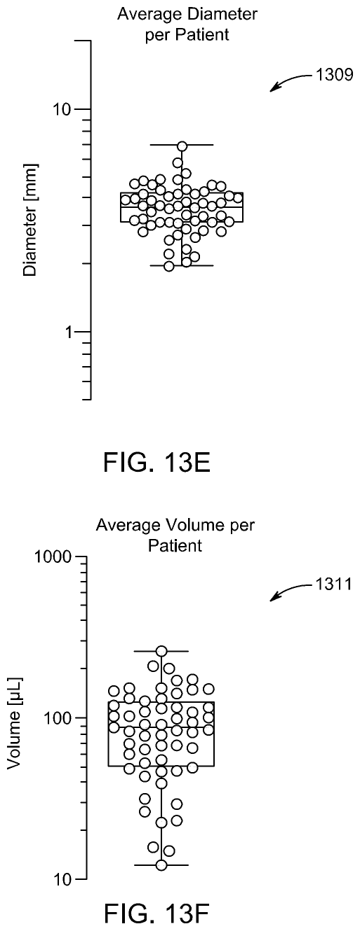

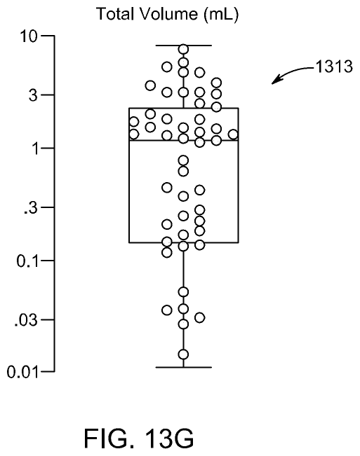

FIGS. 13A-13G illustrate the display of mucus plug characterization data. For example the length of each individual plug 1301, diameter for each determined whole mucus plug 1303, and volume for each whole mucus plug 1305 may be generated using the systems and methods described herein. Additionally average length per patient for whole mucus plugs 1307, average diameter per patient for whole mucus plugs 1309, and the average volume per patient for whole mucus plugs 1311 may be generated. The total whole mucus plug volume 1313 may also be determined by summing all mucus plugs per patient. FIGS. 13A-13G provide a visual representation of the generated data in a logarithmic scale.

FIGS. 14 and 15 are directed to an embodiment of the present disclosure where the annotation of mucus plug portions on the obtained images may be done automatically using a computer-based machine learning algorithm.

FIG. 14 provides an illustration of how the detection of mucus plug portions within a provided radiology image can be automated. As illustrated, in some embodiments, a trained machine learning algorithm may be applied on the received plurality of images to annotate detected mucus plug portions. Image processing algorithms trained on radiologist marked data may be used to detect the mucus plug portions. In some embodiments, the trained machine learning algorithm may include a transformer model that performs segmentation such as a U-Net Transformer.

In some embodiments, the trained machine learning model may include a convolutional neural network. For example, a Mask R-CNN architecture may be used for the machine learning model. The machine learning model may analyze two-dimensional slices. In particular, the CNN may be configured to intake a single slice and output both a bounding box and segmented pixels for likely mucus plugs. Each plug portion may be represented as if it were a separate plug. The results of multiple slices from the same CT scan may then be combined to generate the total score. In some embodiments, the CNN is trained on the segmented pixels of each CT scan.

A machine learning based model may be trained using a labeled data set of annotated and segmented mucus plugs. In some embodiments, the machine learning model may include a deep-learning based automated algorithm that is configured to quantify mucus plug burden. The mucus slice score in particular allows automation in that it is well-suited towards training two-dimensional algorithms. In addition, quantitative three-dimensional metrics can be reconstructed by generalizing the algorithm to the entirety of a chest CT scan.

FIG. 15 illustrates validation studies from using the computer-based machine learning model to generate annotations of mucus plug portions in slices. As illustrated, mucus slice scores from radiologist annotated slices 1501 may be plotted against mucus slice scores obtained from slices with portions marked by a convolutional neural network 1503.

In some embodiments, the disclosed systems and methods may provide a comprehensive suite of quantitative lung mucus biomarkers that can be deployed in research studies that aim to understand the prevalence of mucus phenotypes of lung disease, the relationships between lung mucus pathology and lung function, the molecular mechanism of lung mucus pathology, and the natural history of mucus plug pathology in patients with mucus-associated lung disease. In some embodiments, the systems and methods described herein may be used to generate new treatments, establish treatment plans for patients, and monitor the efficacy of treatments. For example, the location of whole mucus plugs within the airways may help determine which muco-active or anti-inflammatory treatments may be most effective for a subject with a mucus-associated lung disease. Further, quantitative metrics regarding mucus plugs, such as their surface area, or total volume or length, may further guide dosage practices for pharmacological drugs and treatments.

In some embodiments, the comprehensive suite of lung mucus biomarkers that can be deployed as predictive and monitoring biomarkers in clinical trials of muco-active or anti-inflammatory drugs in patients with asthma, COPD, and other mucus-associated lung diseases. In some embodiments, the comprehensive suite of quantitative measurements provided herein may be used for precision medicine. For example, the systems and methods described herein may allow health care providers to identify patients with mucus-high disease phenotypes of the lungs and to target these patient subgroups with muco-active or anti-inflammatory drugs.

In some embodiments, the systems and methods described herein may be used to predict or monitor the efficacy of treatments based on the determined subject-level quantitative assessment of airway mucus path pathology, wherein the treatment includes at least one of a muco-active drug, an anti-inflammatory drug, interventional bronchoscopy, a cough assist device, or a vibrating vest. In some embodiments, the systems and methods described herein may be used to predict or monitor the efficacy of treatments based on the determined subject-level mucus plug phenotype, wherein the treatment comprises at least one of a muco-active drug, an anti-inflammatory drug, interventional bronchoscopy, a cough assist device, or a vibrating vest. For example, mucus plugs determined to be in proximal airway locations that affect lung function may be determined to be treatable by aerosolized drugs or by interventional bronchoscopy.

In some embodiments, the systems and methods described herein can be used to better study airflow obstruction and design muco-active treatments by generating information about the size, airway location, and longitudinal behavior of mucus plugs. For example, the systems and methods described herein can be used to develop specific treatments for mucus plugs such as drugs to decrease the formation of new mucus plugs, drugs to remove existing plugs, or mechanical approaches such as mucus clearance devices or bronchoscopy. The rational development or selection of best treatments to remove mucus plugs requires quantitative data and analysis about their structural features and airway tree location, which can be obtained using the systems and methods described herein.

In some embodiments, the systems and methods described herein can be used in generating one or more visualizations of airways and mucus plugs located within the airway. This may in turn allow for the monitoring of disease progression or treatment effectiveness.

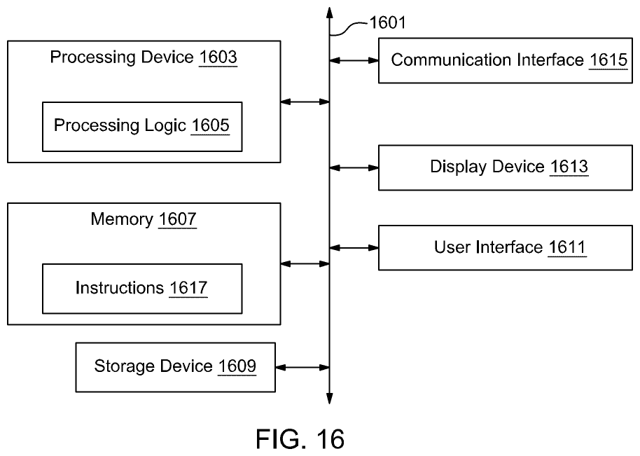

FIG. 16 illustrates a computer system for use in accordance with an embodiment of the present disclosure. FIG. 16 illustrates a functional block diagram of a machine in the example form of computer system 1600, within which a set of instructions for causing the machine to perform any one or more of the methodologies, processes or functions discussed herein may be executed. In some examples, the machine may be connected (e.g., networked) to other machines as described above. The machine may operate in the capacity of a server or a client machine in a client-server network environment, or as a peer machine in a peer-to-peer (or distributed) network environment. The machine may be any special-purpose machine capable of executing a set of instructions (sequential or otherwise) that specify actions to be taken by that machine for performing the functions describe herein. Further, while only a single machine is illustrated, the term “machine” shall also be taken to include any collection of machines that individually or jointly execute a set (or multiple sets) of instructions to perform any one or more of the methodologies discussed herein.

Example computer system 1600 may include processing device 1603, memory 1607, data storage device 1609 and communication interface 1615, which may communicate with each other via data and control bus 1601. In some examples, computer system 1600 may also include display device 1613 and/or user interface 1611. In some embodiments, the user interface 1611 may include a graphical user interface.

Processing device 1603 may include, without being limited to, a microprocessor, a central processing unit, an application specific integrated circuit (ASIC), a field programmable gate array (FPGA), a digital signal processor (DSP) and/or a network processor. Processing device 1601 may be configured to execute processing logic 1605 for performing the operations described herein. In general, processing device 1603 may include any suitable special-purpose processing device specially programmed with processing logic 1605 to perform the operations described herein.

Memory 1607 may include, for example, without being limited to, at least one of a read-only memory (ROM), a random access memory (RAM), a flash memory, a dynamic RAM (DRAM) and a static RAM (SRAM), storing computer-readable instructions 1617 executable by processing device 1603. In general, memory 1607 may include any suitable non-transitory computer readable storage medium storing computer-readable instructions 1617 executable by processing device 1603 for performing the operations described herein. Although one memory device 1607 is illustrated in FIG. 16, in some examples, computer system 1600 may include two or more memory devices (e.g., dynamic memory and static memory).

Computer system 1600 may include communication interface device 1615, for direct communication with other computers (including wired and/or wireless communication), and/or for communication with a network. In some examples, computer system 1600 may include display device 1613 (e.g., a liquid crystal display (LCD), a touch sensitive display, etc.). In some examples, computer system 1600 may include user interface 1611 (e.g., an alphanumeric input device, a cursor control device, etc.).

In some examples, computer system 1600 may include data storage device 1609 storing instructions (e.g., software) for performing any one or more of the functions described herein. Data storage device 1609 may include any suitable non-transitory computer-readable storage medium, including, without being limited to, solid-state memories, optical media and magnetic media.

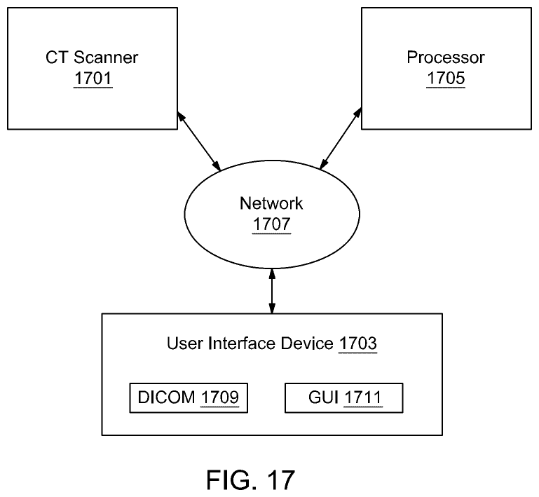

FIG. 17 provides an example system diagram for a quantitative assessment of airway mucus pathology. As illustrated in FIG. 17, in one example, a CT scanner 1701, a user interface device 1703, and a processor 1705 may be communicatively coupled over a network 1707. In some embodiments, the user interface device 1703 may include a DICOM viewer that is configured to display CT scan images to a radiologist and receive annotations for mucus plug segments. The user interface device 1703 may also include a graphical user interface 1711 or other application configured to display visual representations of the quantitative assessments discussed herein such as graphs, charts, medical reports, and the like. Further, the user interface 1711 may also be configured to display an airway mucus map. In some embodiments, the image processing pathways described herein and processes of FIGS. 2 and 3 may be executed by processor 1705 and provided to the user interface device 1703 for display.

### EXPERIMENTAL DATA AND RESULTS

### Experiment #1

Quantitative Analysis of Mucus Plugs in Computed Tomography (CT) Lung Scans from Patients with Severe Asthma Reveals Heterogeneous Mucus Plug Phenotypes and Enables Automation via Deep Learning Approaches

Data from the Severe Asthma Research Program (SARP) study which characterized mucus plugging in patients with severe asthma was used to train a deep learning model. Airway mucus plugs occur in more than half of the participants in the Severe Asthma Research Program-2 (SARP-3) cohort and “mucus plug-high” asthma is characterized by severe airflow obstruction and airway type-2 inflammation. Prior to the application of the systems and methods described herein, phenotypic features of airway mucus plugs (length, diameter, volume) and their location in the airway tree are poorly understood and this lack of this information hampers rational design of muco-active drugs and optimal design of clinical trials of these drugs. Annotating CT lung images manually by skilled radiologists information is time consuming and difficult to scale to large imaging datasets.

### Methods

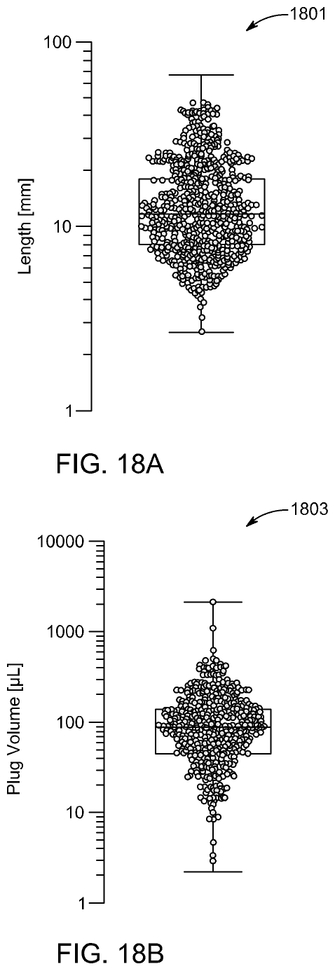

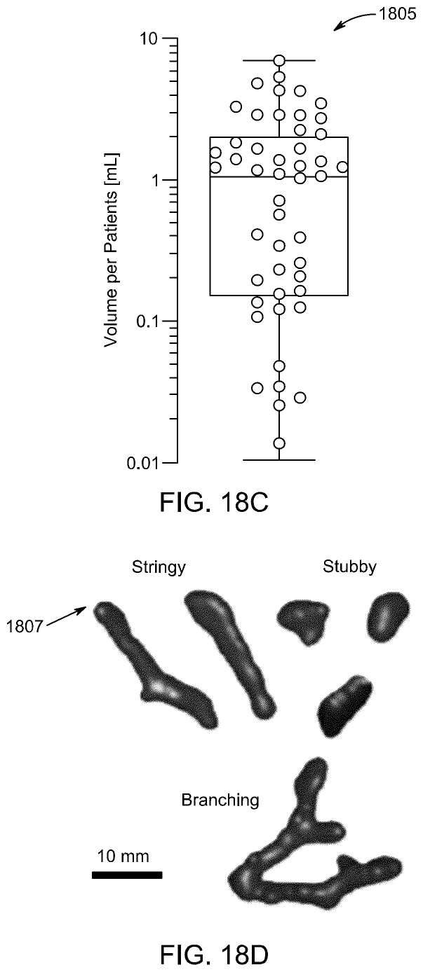

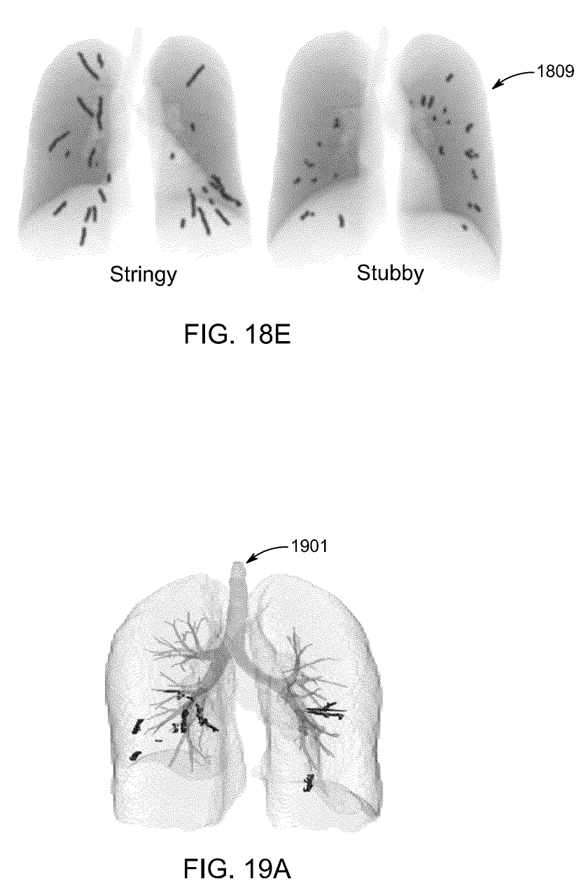

Four radiologists annotated the location and size of 794 mucus plugs in 60 MDCT lung scans (total of 10,990 plug annotations) from asthma patients enrolled in the Severe Asthma Research Program-3 (SARP-3). Scans were selected at random from the subset that were known to have at least 1 mucus plug. A soft-clustering algorithm was used to generate pixelwise annotation from radiologist region-of-interest annotations. Mucus plugs were analyzed for length and volume, and individual plugs were summed to estimate mucus plug volume per patient. For example, data from Experiment #1 is shown in FIGS. 18A-18E. Scatter plots of individual mucus plug lengths 1801, volume 1803, and volume per patient 1805 are shown. Individual plugs were rendered in 3-dimensions 1807 as well as within the entire lung 1809. A 2D convolutional neural network (CNN) was trained on a subset of annotations (27 scans, 6,400 annotations) to automate radiologist annotations.

### Results

Mucus plugs were found to predominantly locate to airway generations 5 to 9 and have a median (IQR) individual length of 11 (7.7-17.4) mm, individual volume of 80 (42-137) μL, and total (within patient) volume of 1.1 (0.2-2.0) mLs. Rendering of mucus plug revealed three phenotypes which can be categorized as “stringy”, “stubby”, and “branched.” Further, these phenotypes tended to be conserved within a patient. Preliminary validation data found good concordance between the 2D CNN algorithm and the radiologist annotations (R²=0.852), analogous to the data illustrated in FIG. 15.

### Conclusion

Mucus plugs in asthma occur mainly in subsegmental airways and are heterogeneous in their shape features. Quantitative mucus plug data can inform rational design of muco-active drugs and provide predictive and monitoring biomarkers for clinical trials of muco-active drugs, anti-inflammatory drugs, interventional bronchoscopy, or vibrating vest-based treatments in mucus plug-high patients with asthma or other mucus-associated airway diseases. The processing pipeline described herein provide deep learning-based approaches to automate mucus plug phenotyping.

### Experiment #2

Mucus Plugs are Associated with Airway Obstruction

Airway mucus plugs occur frequently in participants in the Severe Asthma Research Program (SARP) cohort, and “mucus plug-high” asthma is characterized by severe airflow obstruction. This experiment demonstrated that mucus plugs are mechanistically linked to airflow obstruction and air trapping via direct occlusion of airways, and that the location of mucus-occluded airways determines the severity of obstruction and trapping.

### Methods

Thoracic radiologists performed detailed annotations of airways completely occluded with mucus in chest computed tomography (CT) scans from 55 patients in the SARP cohort. Pixelwise segmentation information for each plug was generated based on the annotations. Mucus plugs were coregistered with the airway tree to generate a patient-specific mucus plug map for each scan. Mucus plug location was used to generate two novel imaging metrics related to airway resistance and airflow obstruction. First, the estimated resistance of the airway tree in the presence of plugging (R^{P}) was divided by the estimated resistance of the native airway tree in the absence of plugs (R^{a}) to yield the relative airway resistance (R^{P}/R^{a}×100) due to plugs. Second, the voxel volume of the lung region associated with a mucus occluded airway (V^{o}) was divided by the total voxel volume in the lobe (V^{t}) to generate the estimated obstructed lung volume percentage (V^{o}/V^{t}×100) per lobe.

### Results

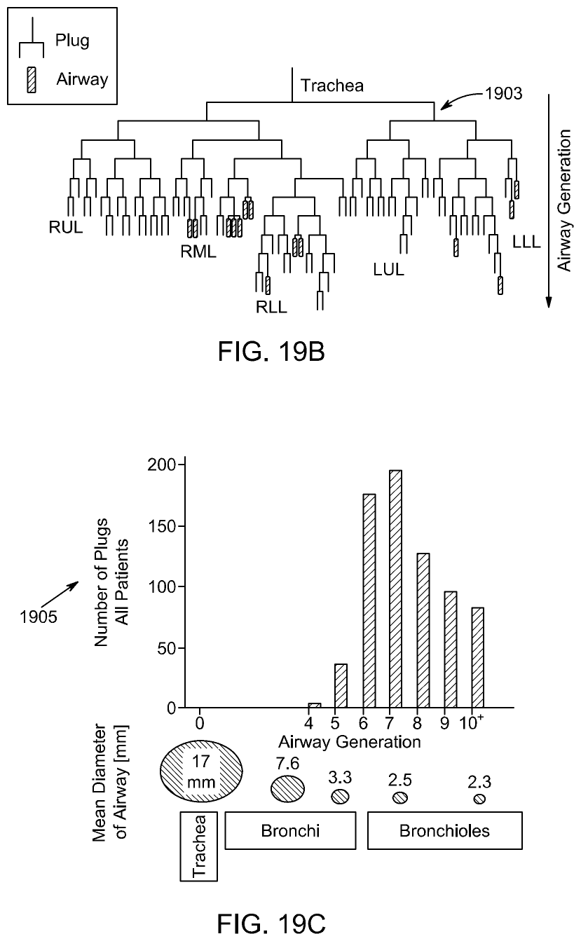

Results from Experiment #2 are shown in FIGS. 19A-19C. A single patient volumetric rendering of mucus plugs, airways, and lung was generated 1901. As shown, segmented mucus plugs may be co-registered with airways. An airway mucus plug map 1903 was derived for the same patient. The airway mucus plug map depicts the topological location of plugs in an airway tree. Among 739 mucus plugs identified in the 55 asthma patients, plugs predominantly localized to airway generations 5 to 10 (2.3-3.3 mm mean diameter, panel C; minimal resolvable airway ˜1.5 mm) 1905. The relative airway resistance (R^{P}/R^{a}) ranged from 100% to 183% and was negatively correlated with a measure of airflow obstruction (FEV1, r=−0.32, p<0.05) while the obstructed lung volume percentage (V^{o}/V^{t}) also was negatively correlated with airflow obstruction (p<0.05).

### Conclusion

CT-evident mucus plugs in asthma are mainly located in 2-4 mm airways and spatially associate with air trapping and airflow obstruction in proportion to their effect on estimated airway resistance. Mucus plugs in the large airways (>2 mm diameter) are mechanistically linked to airflow impairment due to direct occlusion of the conducting airway tree. Accordingly, mucus plugs may form a target to improve airflow obstruction in asthma.

### Experiment #3

### Persistent Mucus Plugs in Proximal Airways are Consequential for Airflow Limitation in Asthma

The systems and methods described herein were used to generate data regarding the size, airway location, and longitudinal behavior of mucus plugs in patients with asthma. The resulting data was used to model airflow obstruction and design muco-active treatments.

### Methods

In accordance with systems and methods described herein, CT lung scans from 57 patients with asthma were analyzed to quantify mucus plug size and airway location, and paired CT scans obtained 3 years apart were analyzed to determine plug behavior over time. Radiologist annotations of mucus plugs were incorporated in an image processing pipeline (such as that described herein) to generate size and location information that was related to measures of airflow.

For this experiment, patient data was obtained from the NHLBI SARP database, a multi-institutional cohort designed to obtain longitudinal clinical, serologic, physiologic, and imaging data of patients with severe asthma. CT scans were acquired after use of a bronchodilator using a previously described protocol. A sample size of 54 was calculated based on an initial power estimate needed to demonstrate an association between mucus plugs in proximal generations and FEV₁. Based on this estimate, 57 patients were selected from a larger cohort of patients whose CT lung scans had previously been scored by radiologists and shown to have mucus plugs. Of the 57 patients, 43 had a second CT lung at year 3. Scans were included in the experimental data if they had at least 1 mucus plug either at baseline or year 3. In total, at the baseline visit, CT scans from 55 patients had mucus plugs that were analyzed and included in the baseline data set; at the year-3 visit, CT scans from 42 patients had mucus plugs that were analyzed and included in the year-3 data set. Scans were excluded if they demonstrated radiographic evidence of active infections, allergic broncho-pulmonary aspergillosis, lung scarring, or motion degradation limiting the ability to evaluate for mucus plugs. All eligible SARP-3 scans acquired at the UCSF center were included in the study, and additional scans were randomly sampled from the remainder of the SARP-3 CT lung imaging database.

Thoracic radiologists annotated the chest CTs in this experiment. Readers used a DICOM viewer (OsiriX; Pixmeo) to place an elliptical marking over each mucus plug within an axial slice with a window width 1,200 HU and window center was HU during visualization. Voxel spacing of the reconstructed volumes ranged from 0.5 to 0.7 mm in the axial (x and y axes) plane, and spacing between axial slices (z axis) ranged from 0.5 to 0.6 mm. Each annotation yielded a center coordinate, width, and height for a region of interest (ROI) containing the plug at that slice. This process was repeated for every plug and every axial slice in the scan. Annotations that belonged to a single contiguous plug were designated with a single numerical label.

The annotation process was performed independently twice by 2 radiologists for each scan. Plugs that were identified by only 1 of 2 readers were considered discordant and reviewed by a third reader for adjudication. From the finalized annotation, the mucus segment score was calculated after manual identification of the bronchopulmonary segment containing each mucus plug. The mucus slice score was calculated as the sum of the number of elliptical annotations, and mucus plug score was calculated as the sum of the number of individual mucus plugs.

Annotations of the year-3 scans occurred after the baseline scans, and the radiologists had access to the finalized baseline scans results during annotation. Similar to the baseline scan process, two radiologists independently annotated each year-3 scan, followed by adjudication by a third radiologist. In certain cases, the annotator of the year-3 scan identified a likely plug on the baseline scan that had not been annotated during the initial process. These possible baseline plugs were collectively reviewed by the entire team of four radiologists, and a consensus vote was taken to determine if the plug should be retroactively annotated on the baseline scan. In this manner, an additional 34 plugs in the baseline cohort were identified and annotated. This consensus read was undertaken to obtain higher fidelity data in mucus plug tracking and in identifying mucus plug persistence over time.

To segment and analyze individual plugs, systems and methods in accordance with the description herein were used to ingest and process annotations. Each annotation was first used to extract an elliptical ROI surrounding each mucus plug in a particular slice. The extracted voxels from all slices belonging to a single mucus plug were combined into a single volumetric subset. A fuzzy clustering algorithm known as Gustafson-Kessel (GK) clustering was used to segment the mucus plug from surrounding lung parenchyma and airway lumen in a manner similar to that described for segmentation of lung nodules. The GK clustering algorithm was run on the extracted volumetric subset and was used to separate voxels into 2 clusters based on imaging intensity (radiodensity). The foreground was taken to be the cluster with the highest intensity value. The single largest contiguous foreground region by volume was then selected as the mucus plug.

Once individual mucus plugs were segmented on a volumetric basis, their size was estimated using voxel and mesh-based methods. The length of each plug was computed by employing principal component analysis on the ROT to calculate eigenvalues along the 3 principal axes (λmaj>λₘᵢₙ>λleast) and estimating the length L by the following formula: L=4√λₘₐⱼ. The diameter was calculated by fitting the 3D mucus ROT to a cylinder and using the resultant best-fit value for the cylinder diameter. The CT radiodensity of segmented pixels was analyzed per plug to compute the median density value for each individual plug. For visualization of individual mucus plugs, a triangular mesh representing the surface of the mucus plugs was generated using the marching cubes algorithm with an additional surface smoothing algorithm applied prior to rendering.

Lung parenchyma was segmented on a lobar basis. Airway segmentation was performed by combining a region-growing method, which yields an estimate of central airways, with a convolutional neural network-based approach, which has improved performance in smaller airways. The segmented airway was taken to be the largest contiguous region resulting from the voxel-wise union of the two methods. The airway tree was then skeletonized yielding a centerline estimation of the airway tree. A topological representation of the airway tree was generated that contained information for each portion of the airway tree, including the location of centerline points, branching points, length of each segment, local airway radius estimates, airway generation number, and lobar location as well as information about connectivity to more distal (child) branches. Airway termination points were defined as the most distal points of the centerline that no longer had child branches.

After individual mucus plugs were segmented, each plug was then localized to a position in the airway tree. For each mucus plug, a search was performed for the nearest airway termination point by Euclidean distance. Mucus plugs were then incorporated into the topological diagram of the airway tree. The lobe of each plug was assigned based on the lobe of the airway to which it localized, and the generation was computed by counting the number of airway bifurcations from the trachea, with the trachea considered generation 0.

To estimate the total effect of mucus plugs on airflow obstruction, the information generated in the airway mucus plug map for each CT scan was used to calculate a measure of airway resistance (RS). First, the total airflow Q through the visible airway tree due to an applied pressure Δ/F was computed by converting the airway tree into a network of resistive elements. The length Lₙ and radius rₙ of each airway segment n was used to estimate the resistance Rₙ through that portion of the airway using Rₙ=8 μLₙ/πrₙ⁴, where μ is the dynamic viscosity of humidified air. The resulting series of flow and pressure equations at each node was then solved. Of note, the formula for Rₙ reflects Poiseuille flow. Even if certain assumptions of Poiseuille flow are violated, the resistance of airway segments in the lung is still inversely proportional to the fourth power of airway radius. The effective resistance R^{α} of the entire tree in the absence of mucus plugs was calculated as R^{α}=ΔF/Q^{α}. In the next step, each terminal branch of the airway tree was considered to be obstructed by associated mucus plugs, as determined by the airway mucus plug map. Rp was computed as the net resistance with these branches blocked (i.e., flow is set at zero at those nodes). RS was then calculated as the percentage increase in resistance due to plugs above the unplugged airway by RS=(100×[Rp−R^{α}]/R^{α}). RS was not able to estimated in 3 of 97 scans (3%) because the processing pipeline did not converge on the parameter estimates for the entire airway. The Pearson correlation coefficient for R^{α} between baseline and year-3 scans was 0.72.

After lobar segmentation, the voxels within each lobe were assigned to a specific airway branch by finding the nearest airway termination point. Each subregion was then labeled as obstructed if a mucus plug occluded the terminal airway and unobstructed if a mucus plug was absent. The OLVP for each lobe was estimated as the volume of voxels associated with an obstructed airway) (V^{o}) divided by the total voxel volume of the lobe (V^{t}), or OLVP=(100×V^{o}/V^{t}). The computation was performed on a lobar basis to ensure that lung parenchyma was not assigned to an airway branch opposite a fissure, after which the OLVP was then estimated for the entire lung. OLVP could not be computed in 3 of 97 scans (3%) where lobar segmentation failed.

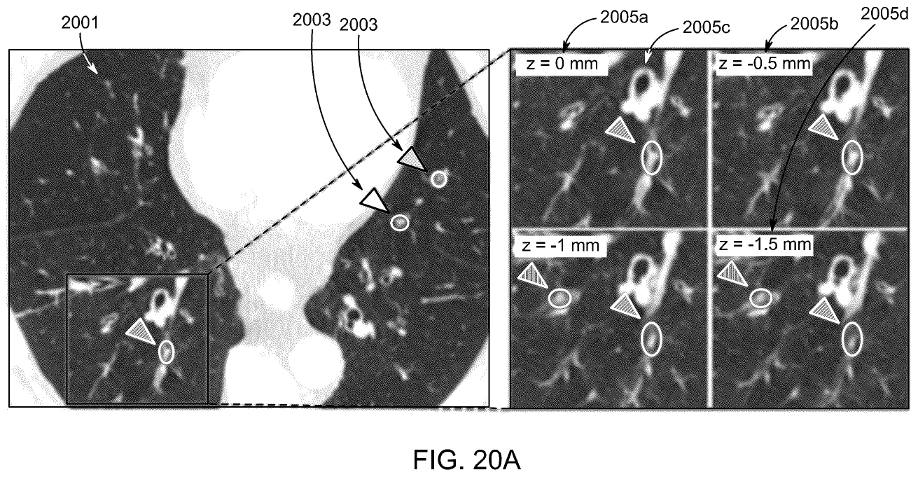

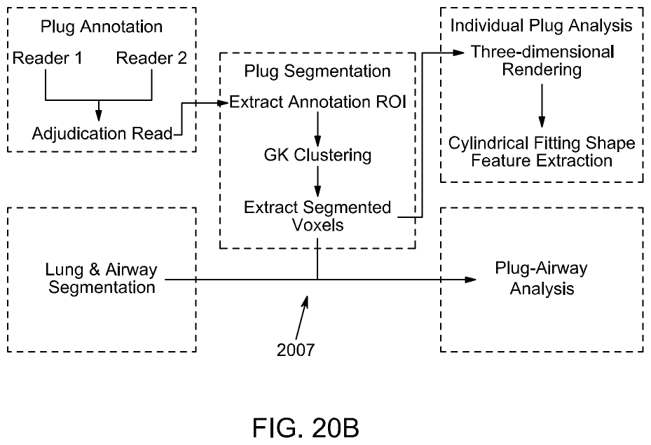

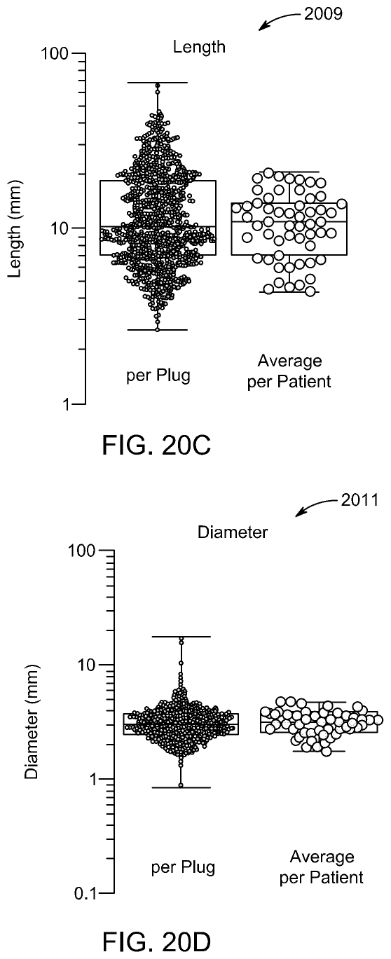

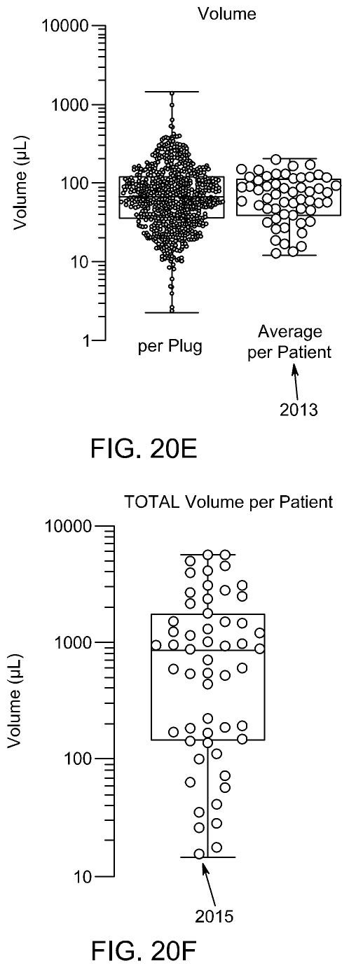

As illustrated in FIGS. 20A-20F, experimental data indicates that mucus plugs appear heterogenous in size and shape for patients with asthma. An overview of an annotation and image processing platform built in accordance with the description herein illustrates quantification of mucus plug shapes. As shown in 2001 an elliptical mark can be placed over each plug and generate a center coordinate, width, and height for region of interest 2003 (as depicted by arrows). The process can be repeated at each axial slice 2005a, 2005b, 2005c, 2005d, with z indicating the slice location relative to the initial image. Annotations for the same plug can be indicated with color, a symbol, a label, or the like. As shown, annotations can be incorporated into an image processing system for segmentation and feature extraction on plugs, enabling calculation of their length, diameter, and volume 2007. Results of shape feature quantification of individual mucus plugs (left, n=778 plugs) and averages by patient (right, n=55 patients) from baseline scans, including plug length 2009, plug diameter 2011, and plug volume 2013 are shown. Note that scales are logarithmic. Bars indicated interquartile range, and whiskers show minimum and maximum values. Total mucus volume per patient 2015 is also illustrated.

Automated quantitative CT analysis was performed by Vida Diagnostics to estimate DPM air trapping, Jacobian mean, and LAA⁸⁵⁶% on a lobar level.

Clinical surveys of asthma control, comorbid conditions, spirometry, hematologic testing, and sputum characterization were collected and analyzed as part of the SARP-3 protocol. Values were taken from the visit closest to the date of the designated CT scan. Not all patients had data for every study outcome, and analyses used available data.

Statistical analyses were carried out using the SciPy, scikit-learn, and statsmodel packages in Python. Numeric nonparametric variables were evaluated by nonparametric methods including Kruskal-Wallis, Mann-Whitney U, or Wilcoxon signed-rank test (matched samples). Categorical variables were evaluated by χ² analysis. Regression of numeric variables was quantified using the Spearman correlation coefficient (rₛ). For linear regressions on variables with repeated measurements from the same patient, F values were additionally confirmed using a linear mixed model with random effects for patient. A F value of less than 0.05 was considered significant. For analysis of proximal versus distal airway mucus plugs, confidence intervals for rₛ of plug count by generation versus FEV₁ and FEF25-75 were obtained by bootstrapping. In each bootstrapping sample, a set of 55 patients was generated using random resampling with replacement. The process was repeated 1,000 times. Statistical significance in comparing rₛ for generation ≤7 and generation ≥10 was determined by estimating the 95 (P<0.05) or 99 (P<0.01) percentile value of the quantity (rsgen≤7−rsgen≥10) from the bootstrap distribution. SHAP value analysis was carried out using the SHAP Python package. Directed acyclic graph analysis was performed using DAGitty.

### Results

The length distribution of 778 annotated mucus plugs was multimodal, and a 12 mm length defined short (“stubby”, ≤12 mm) and long (“stringy”, >12 mm) plug phenotypes. High mucus plug burden was disproportionately attributable to stringy mucus plugs. Mucus plugs localized predominantly to airway generations 6-9, and 47% of plugs in baseline scans persisted in the same airway for 3 years and fluctuated in length and volume. Mucus plugs in larger proximal generations had greater effects on spirometry measures than plugs in smaller distal generations, and a model of airflow that estimates the increased airway resistance attributable to plugs predicted a greater effect for proximal generations and more numerous mucus plugs.

Experimental data indicates that annotations of mucus plugs in CT lung scans provide measures of airway mucus plug burden. A mucus plug “segment score” was generated when a radiologist assigns a point to each bronchopulmonary segment in a CT lung scan that has at least 1 airway occluded by mucus. The segment score has a limited range of values (values 1-20), is not fully quantitative, and does not provide information about the airway location of a mucus plug or its shape and size features. To address these limitations and answer research questions related to mucus plug characterization, annotators (thoracic radiologists) can use a Digital Imaging and Communications in Medicine (DICOM) viewer to place elliptical markings on airways occluded by mucus in 2D axial slices of CT lung scans. A clustering algorithm can be applied to these elliptical annotations such that the plugs are segmented, reconstructed in 3D, and enumerated. In this way, thoracic radiologists generated 12,476 unique annotations related to 778 individual whole mucus plugs in CT scans from 57 patients with asthma. Clinical characteristics of the patients for which the data was obtained include Age (yr), mean (SD) 51.2±15.2, BMI (kg/m2), mean (SD) 31.9 ±8.4, Race, n (%) 6 (10.5%), Asian, Black/African American 9 (15.8%) White 38 (66.7%), More than 1 race 4 (7.0%), Female, n (%) 38 (66.7%), Severe Asthma A,n (%) 51 (89.5%), ACT, median (IQR) 15, (13, 20), High dose ICS usage, n (%) 53 (94.6%), Daily oral corticosteroid, n (%) 8 (14.3%), Pre-BD FEV1 (% pred), mean (SD) 63.6±17.8, Pre-BD FVC (% pred), mean (SD) 79.8±17.0, Pre-BD FEV1/FVC (%), mean (SD) 63.6±9.3, and IgE (kU/L), median (IQR) 175 (71, 319), and Blood eosinophil count (cells/μL), median (IQR) 327 (181,535).

A point for each elliptical annotation within a patient's CT lung scan was assigned and a patient-specific “mucus slice score” were calculated from the sum of these points. The mucus slice scores were correlated with the mucus plug segment scores and provide more quantitative information and a larger range of values. The total number of discrete mucus plugs per patient is denoted as the “mucus plug score,” and is another total mucus plug burden score with similar advantages.

Experimental data indicates that mucus plugs were found to be heterogeneous in size and cluster into “stubby” and “stringy” phenotypes. To quantify the shapes and sizes of mucus plugs, the voxels for each mucus plug were extracted, and the size of each plug was computed and quantified. The length, diameter, and volume of individual mucus plugs varied across 1 or more orders of magnitude, indicating a high degree of heterogeneity in the size of mucus plugs in asthma. The volume of mucus plugs within each patient was quantified by a total mucus volume measure, which also varied by multiple orders of magnitude. The distribution of mucus plug lengths appeared to be multimodal, and assessment of model fit by Akaike information criterion revealed that a Gaussian mixture model with 3 underlying distributions had the highest likelihood. Based on a length of 12 mm separating the 2 dominant populations in the model, 2 plug phenotypes were defined based on length-short plugs that were 12 mm or less in length, denoted as “stubby,” and long plugs that were more than 12 mm in length, denoted as “stringy.” Among 778 plugs, 448 were categorized as stubby and 330 were categorized as stringy. Information on the numbers of stubby and stringy mucus plugs within each patient allowed determination of the mucus plug volume in each patient attributable to stubby versus stringy plugs. Patients with the highest total mucus volumes achieved these levels mainly because of volume contributed by stringy mucus plugs.

Eosinophilic inflammation, including eosinophil counts and levels of eosinophil peroxidase (EPX) in blood and sputum, are known to be linked to mucus plug segment scores in asthma. Accordingly, the mucus plug data can be used to determine whether the size of individual mucus plugs was influenced by eosinophilic inflammation. Indeed, the average mucus plug length and volume in patients were positively correlated with blood eosinophil counts and sputum EPX levels.

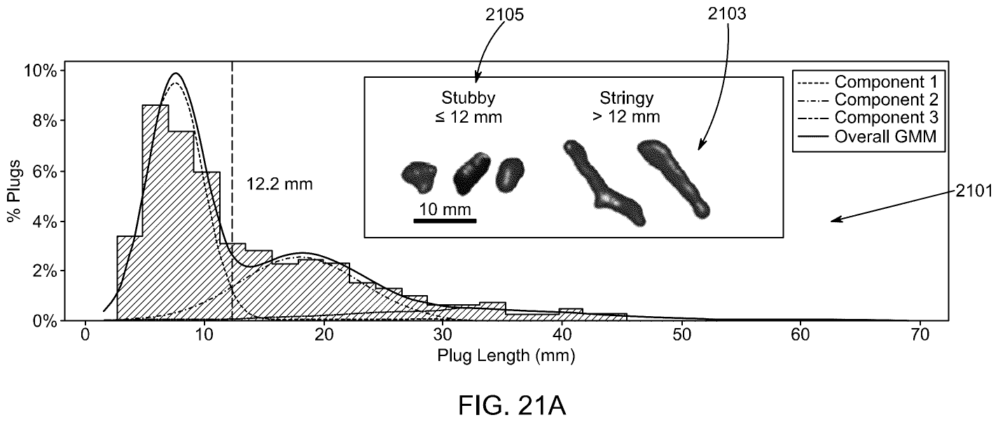

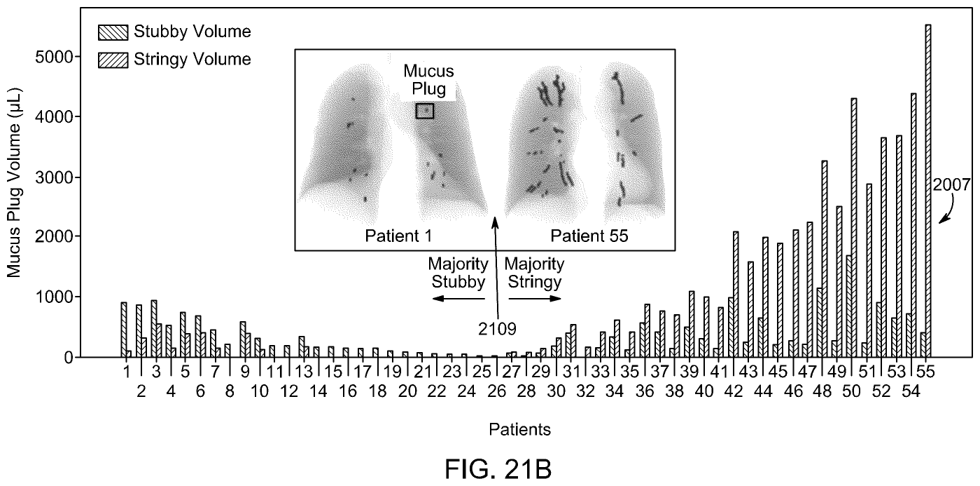

FIGS. 21A-21B also illustrate experimental data indicative of mucus plugs exhibiting multiple underlying length phenotypes. In particular, FIG. 21A illustrates a distribution of mucus plug lengths with best-fit Gaussian mixture model by Akaike information criterion, with separation between short (“stubby”) plugs and long (“stringy”) plugs at 12 mm 2101. As depicted in the chart, very long plugs (component 3) make up a small portion of total population. The inset shows 3D renderings of stubby 2105 and stringy 2103 mucus plugs. FIG. 21B illustrates the distribution of mucus plug volume in each patient (n=55) ordered by predominance of stubby versus stringy mucus plugs within each patient 2107. The inset image provides renderings of mucus plugs within the lung of a patient with a majority stubby plug volume (patient 1) and majority stringy plug volume (patient 55) 2109.

Experimental data indicates that mucus plugs in CT lung images primarily localize to airways that are 2-4 mm in diameter for patients having asthma. By segmenting lung parenchyma and airways on a lobar basis, every mucus plug could be localized to a specific airway branch and lobe. This information allowed the creation of a patient-specific “airway mucus plug map,” a visualization of the location of each mucus plug within the branching airway tree. To summarize the airway generations occluded by all 778 mucus plugs, a frequency distribution plot was generated that shows that mucus plugs are located primarily in generations 6, 7, 8, and 9. These airways are estimated to be typically 2-4 mm in diameter in the CT lung scans analyzed. Although the number of mucus plugs did not differ significantly in upper versus lower lobes or in the right versus the left lung, the volumes of individual mucus plugs in the lower lobes were greater than the volumes of individual mucus plugs in the upper and middle lobes.

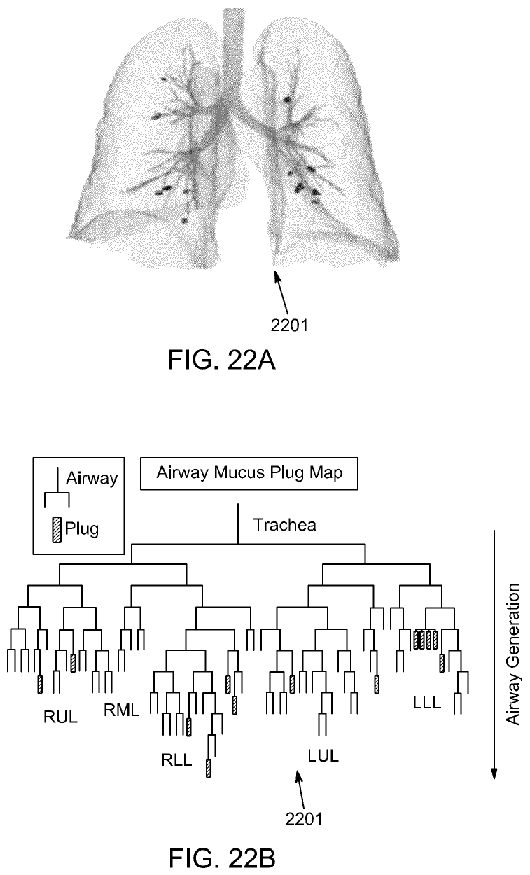

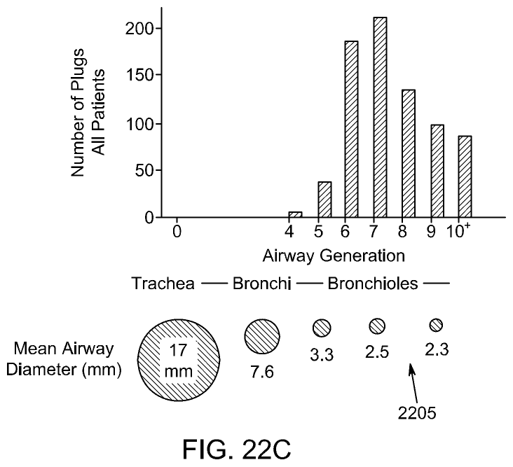

FIGS. 22A-22C illustrate experimental data indicative of the mucus plugs being primarily located in proximal airway generations in patients with asthma. FIG. 22A provides a rendering 2201 of segmented lung parenchyma, airways, and mucus plugs (red) in a patient with asthma. As illustrated in FIG. 22B, a mucus plug map 2203 generated in accordance with the systems and methods described herein shows topological location of mucus plugs in the airway for the same patient. Generation number is counted by each airway bifurcation with trachea as generation 0. Also illustrated is a histogram showing that mucus plugs are located primarily in airway generations 6-9, which have a diameter of 2-4 mm 2205. The experimental data for FIG. 22C are from 778 plugs visible in 55 baseline CT lung scans, and the mean airway diameter is the average diameter measured at each airway generation across the 55 scans.

Experimental data indicates that mucus plugs persist in the same airways for many years but demonstrate dynamic changes in size over time. Of the 57 patients whose baseline CT lung scans were annotated, 43 had a second CT lung scan available at their year-3 visit that allowed analysis of mucus plugs over time. Among scans from the 43 patients, 580 mucus plugs were visible on the baseline scans and 619 mucus plugs were visible on the year-3 scans. The per-patient average plug length, average plug volume, and total mucus plug volume did not differ significantly between baseline and year 3 indicating overall stability of total mucus plug burden within patients over 3 years. The temporal dynamics of the 580 mucus plugs identified in the baseline scans from the 43 patients, were determined by tracking mucus plugs that persisted in the same airway between the baseline and year-3 scans, and labeling these plugs as “persistent.” Mucus plugs that disappeared between the baseline and year-3 scans were determined and labeled as “transient.” 47% of the 580 baseline plugs persisted in the same airway for 3 years, and 81% of the 43 patients had at least 1 persistent plug. Persistent mucus plugs, although static in location, exhibited dynamic behavior in size and underwent variable changes in length and volume. Changes were centered around zero and appeared normally distributed, and there was no statistically significant difference in average length or volume of the entire population of plugs over the 3-year period. In addition, the finding that the total mucus volume per patient stayed, on average, constant over time is consistent with the observation that the disappearance of transient mucus plugs sometimes coincided with the appearance of new mucus plugs in different airways at year 3.

In comparing the characteristics of persistent and transient mucus plugs, the persistent mucus plugs were longer, more frequently stringy, and more frequently located in the upper lobes. CT attenuation of the pixels in each plug were determined by computing the median value in Hounsfield units (HU) and transient plugs were found to be more radiodense. The 3-year behavior of stringy versus stubby plugs using Sankey plot and state-transition analyses indicated that among plugs that persisted, stubby plugs were more likely to stay stubby and stringy plugs were more likely to stay stringy.

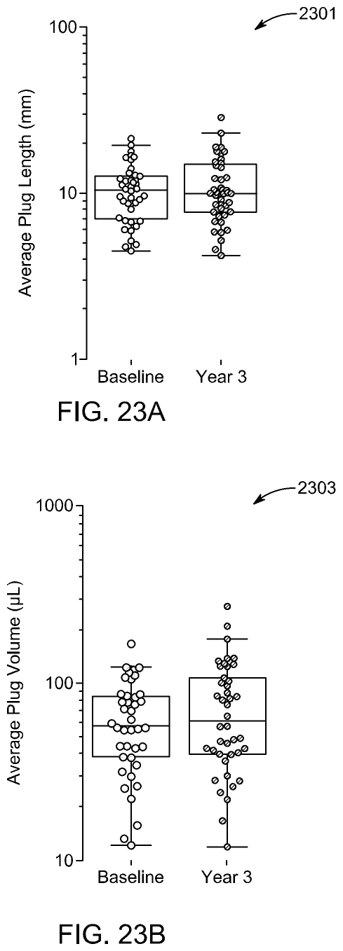

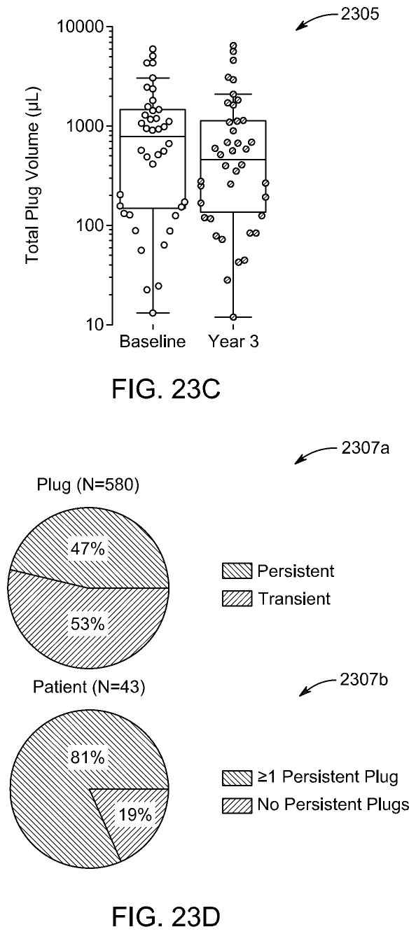

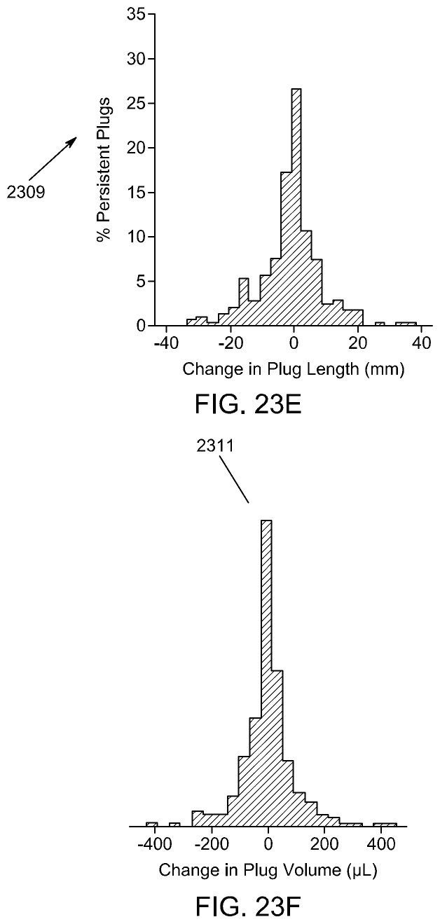

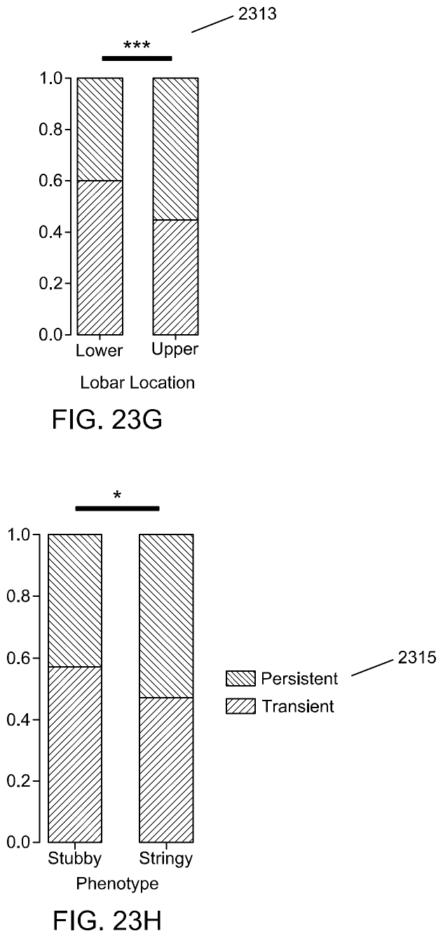

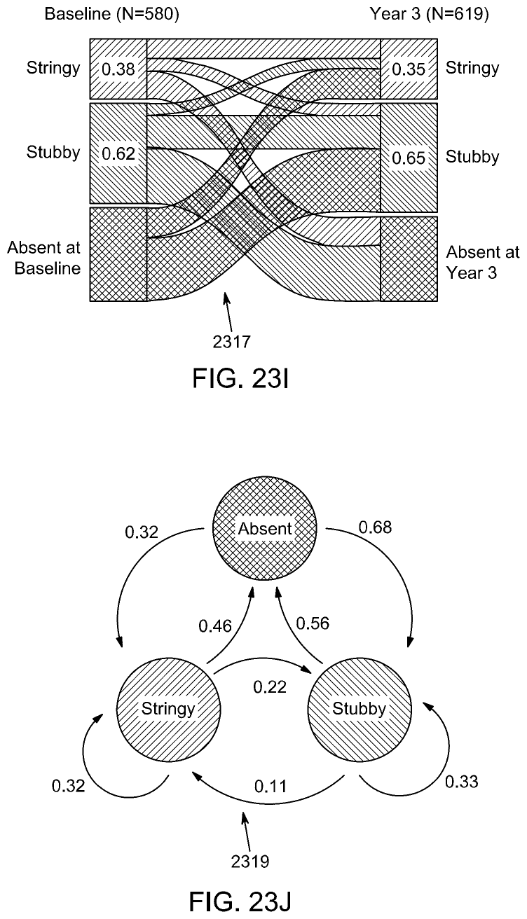

FIGS. 23A-23J provides illustrations characterizing additional experimental results. In particular, FIG. 23D depicts that mucus plugs persist for years in the same airways and demonstrate dynamic changes in size over time. In particular, the illustrations provide a comparison of patient-level measurements for patients with matched baseline and year-3 scans with average plug length per patient 2301, average plug volume per patient 2303, and total plug volume per patient 2305 all showing similar between baseline and year 3. Data for the illustrated comparisons between baseline values and year-3 values are from 580 baseline plugs and 619 year-3 plugs visible in 86 CT lung scans from 43 patients. Bars indicate interquartile range (IQR), and whiskers indicate 1.5 IQR. The dynamic data was analyzed to determine the percentage of persistent plugs. In particular, the percentage of individual plugs classified as persistent or transient using analyses of scans at baseline and year 3 is provided 2307a. Also illustrated is the percentage of patients with at least 1 persistent plug in the same airway at baseline and at year 3 2307b. A frequency distribution plot shows the change in mucus plug length 2309 from baseline to year 3 as well as a frequency distribution plot showing the change in mucus plug volume 2311 from baseline to year 3 are also shown. Data for the frequency distribution plots 2309, 2311 are from 270 persistent mucus plugs. As illustrated in FIGS. 23G-23H, compared with mucus plugs in lower lobe locations, mucus plugs in upper lobe locations are more likely to persist for 3 years 2313 ***P21 0.001 (Kruskal-Wallis test). Additionally, as compared with stubby plugs, stringy mucus plugs are more likely to persist for 3 years 2315. *P<0.05 (Kruskal-Wallis test). Also shown is a Sankey plot 2317 depicting how stringy, stubby, and absent mucus plug phenotypes vary from baseline to year 3. Further, FIG. 23J also provides a state-transition diagram 2319 showing the probability of transition between stubby, stringy, and absent plug group from baseline to year 3.

Experimental data indicates that mucus plugs in proximal airways have larger effects on spirometric measures of lung function than plugs in distal airways. Overall mucus plug burden as assessed by mucus segment score, mucus plug score, and mucus slice score was inversely associated with forced expiratory volume in 1 second (FEV₁). Localization of mucus plugging to specific airway branches, however, allowed comparison of the relative effects of mucus plugs in proximal airways (generations 7 or less), intermediate airways (generations 8 and 9), and distal airways (generation 10 and greater). Correlation coefficients and SHapley Additive explanation (SHAP) values (which consider plug count in each generation as an independent feature in a linear regression) can be determined in order to compare the relative effects of mucus plug count in proximal, intermediate, and distal airways on spirometric measures of airflow. In these analyses, the mucus plugs are grouped independently by airway generation for each patient, and the number of mucus plugs per generation is counted for each patient. The plug count by generation is correlated with spirometry, either the postbronchodilator FEV₁ or the forced expiratory flow between 25% and 75% of forced vital capacity (FEF₂₅₋₇₅), to estimate a Spearman coefficient. For these analyses, the CT scans and lung physiology data from the baseline and year-3 visits are pooled so that 97 CT scans from 57 patients are analyzed. The correlation coefficients (rₛ) for mucus plugs in proximal airways (generation ≤7) and FEV₁ or FEF₂₅₋₇₅ are more negative than the coefficients for mucus plugs and FEV₁ or FEF₂₅₋₇₅ in distal airways (generation ≥10), indicating a stronger negative effect of those plugs on airflow. In addition, the magnitude of SHAP values for mucus plugs in proximal airways was larger than those in distal airways, also indicating a stronger effect from proximal plugs.

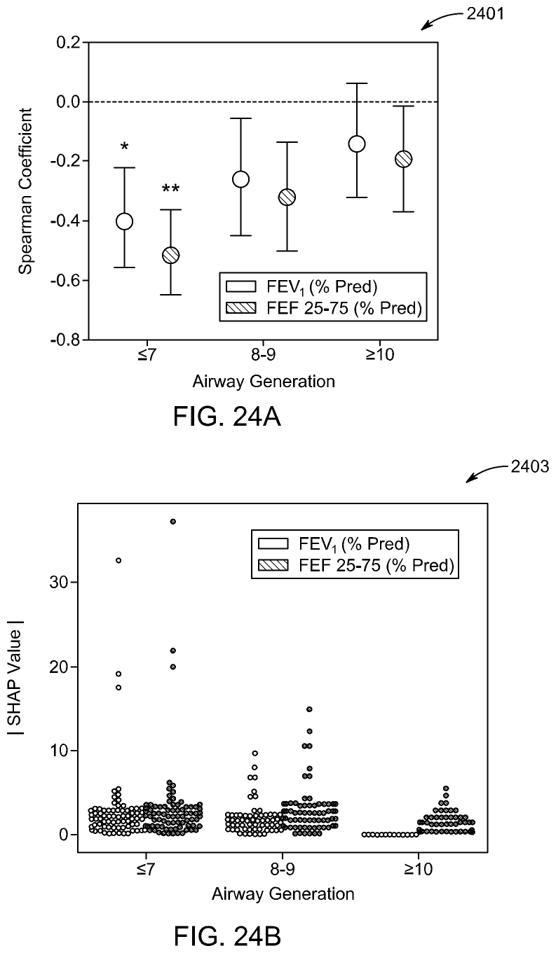

FIGS. 24A-24B provide additional illustrations of experimental data. Illustrated is analytical data indicating that proximal plugs are more consequential for spirometric measures of airflow obstruction in asthma. FIGS. 24A-24B provide correlation analysis of mucus plugs grouped independently by proximal airway generations (7 or less), intermediate airway generations (8 and 9), and distal airway generations (10 and greater) as well as spirometry measures. The estimated Spearman coefficients for each generation group 2401 which correlate plug count by generation group with FEV1 and FEF25-75, are shown for proximal, intermediate, and distal airway generations along with 95% CI. *P<0.05, different from distal airway generations for FEV1 (by bootstrapping). **P<0.01, different from distal airway generation (by bootstrapping). Data presented includes pooled baseline and year-3 follow-up scans (n=97). Also shown are Absolute SHAP values for FEV1 and FEF25-75 at proximal, intermediate, and distal airway generations (n=97) 2403.

Experimental data indicates that mucus plugs are associated with airway-specific increases in resistance score and air trapping. Mucus plugs occlude airways, causing airflow obstruction in the conducting airway tree and air trapping in the lung parenchyma distal to affected airways. Simplified models of airflow and air trapping built in accordance with the systems and methods described herein demonstrate that mucus plugs can obstructing airflow in plugged airways. These patient-specific models intake the segmented airways, lungs, and mucus plugs for each individual CT scan and output 2 measures: (a) the resistance score (RS), an estimated effect on the large airway resistance due to mucus plugs, and (b) the obstructed lung volume percentage (OLVP), an estimate of percentage of lung parenchyma distal to airways occluded by mucus plugs and likely to exhibit air trapping. Consistent with wide variation in total mucus plug burden between patients, RS and OLVP values also varied widely between patients. In cross-sectional analyses of data from the baseline CT lung scans, both values showed significant inverse associations with FEV₁ and FEF₂₅₋₇₅. In addition, the changes in RS and in OLVP from baseline to year 3 correlated with changes in FEV₁ and FEF₂₅₋₇₅. A sensitivity analysis can be performed to determine the effects of an outlier with ΔRS of 201 and ΔFEV₁ of −14%. rₛ was determined to be −0.50 (P=0.001) with this outlier included and −0.46 (P=0.003) with the outlier excluded.

The air trapping model posits that air trapping is spatially associated with occluded airway branches. Lung lobe-specific data for OLVP was generated and analyzed the relationship between OLVP and the disease probability measure of functional small airway disease (DPM-fSAD), a previously described measure of air trapping. DPM-fSAD is quantified from CT lung scans by registering images acquired at inspiration to images acquired at expiration and, on a voxel-by-voxel basis, identifying regions of the lung that trap gas. Lobe-specific OLVP measures correlated significantly with fSAD at baseline and that the change in lobe-specific OLVP from baseline to year 3 correlated with changes in fSAD. OLVP also significantly correlated with two other CT-based functional measures related to air trapping, (a) the Jacobian mean (the inspiratory to expiratory local lung volume ratio) and (b) expiratory low attenuation area percent below −856 HU (LAA⁸⁵⁶%), on a lobar basis. Analysis from linear mixed-effects models to control for multiple measurements from the same patient as well as multivariate regression controlling for age, BMI, sex, and airway wall thickness (covariates determined by a directed acyclic graph) were consistent with these results. In particular, all measures relating OLVP to measures of airflow and air trapping remained statistically significant when controlling for all covariates. Taken together, these data support the interpretation that mucus plugs specifically cause air trapping in the lung region distal to the airways they occlude.

RS is used to further test if mucus plugs located in more proximal locations are more consequential for airflow obstruction. For this analysis, the RS in each patient is calculated and divided by mucus plug score (i.e., plug count) to estimate RS per plug as a measure of each individual plug's effect on airflow obstruction. Mucus plugs can be stratified by proximal (generation ≤7), intermediate (generation 8-9), and distal (generation ≥10) airway generation and found that plugs in proximal generations had significantly higher RS per plug than intermediate or distal generations. Plugs from patients with high and low mucus plug scores can also be stratified based on the median value of baseline patients, 11 plugs. Plugs in patients with high mucus plug scores had a higher RS-per-plug score, consistent with the interpretation that, as mucus plugs begin to occlude a substantial fraction of large airways and leave fewer airways patent, subsequent mucus plugs have a higher marginal effect on net airway resistance. These data indicate that more numerous mucus plugs in more proximal locations are more consequential for airflow obstruction and air trapping than sparser and more distal mucus plugs.

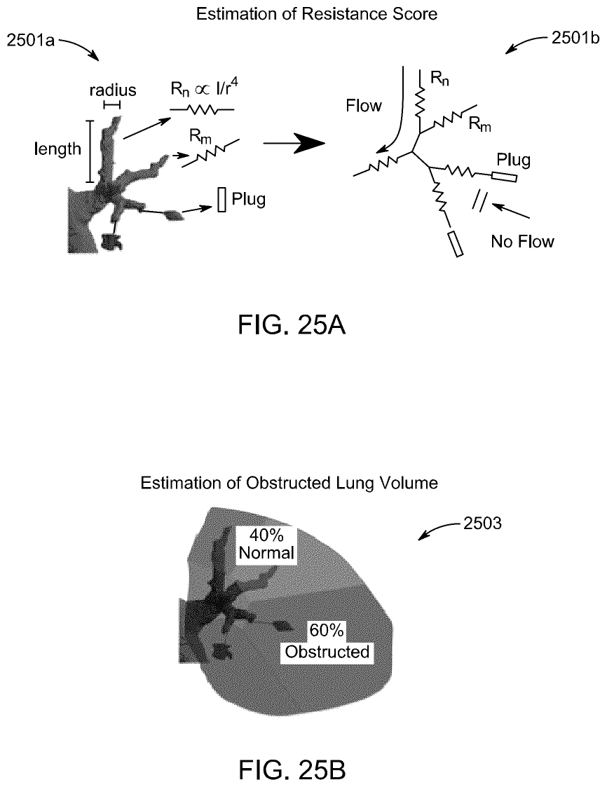

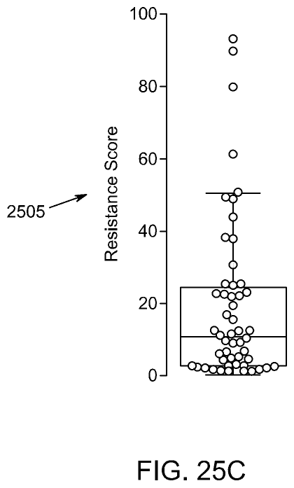

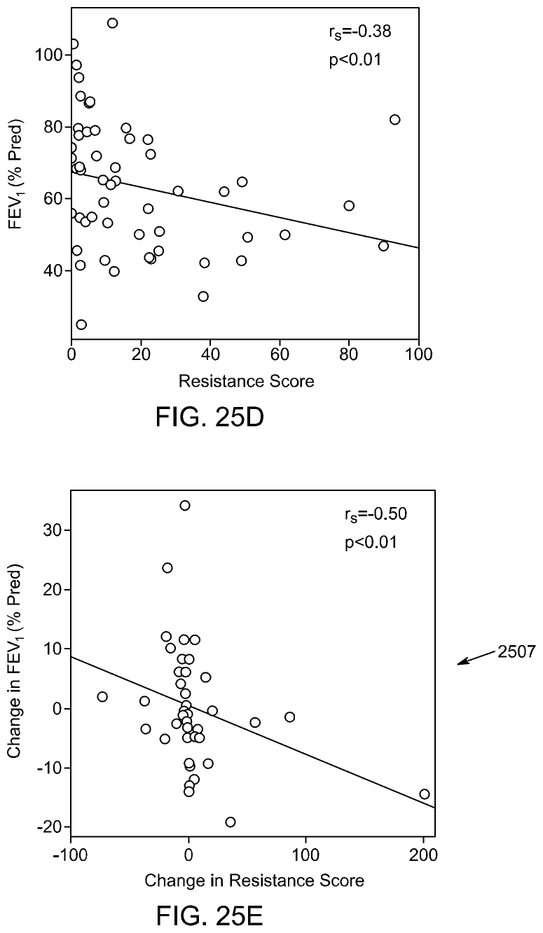

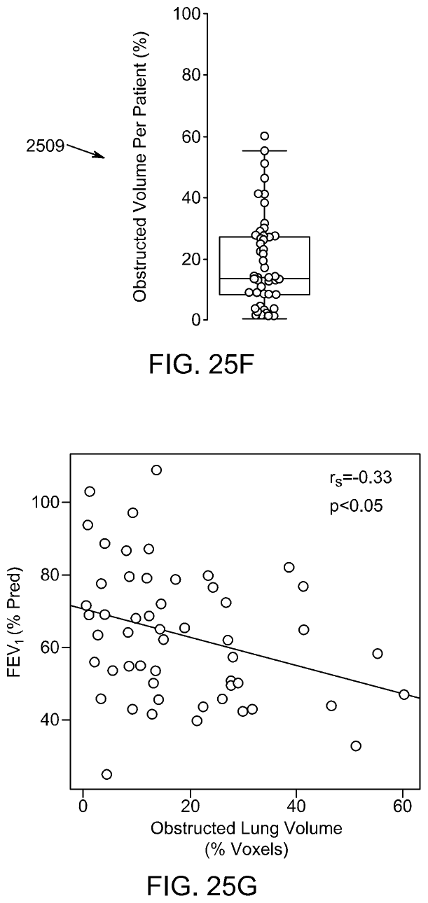

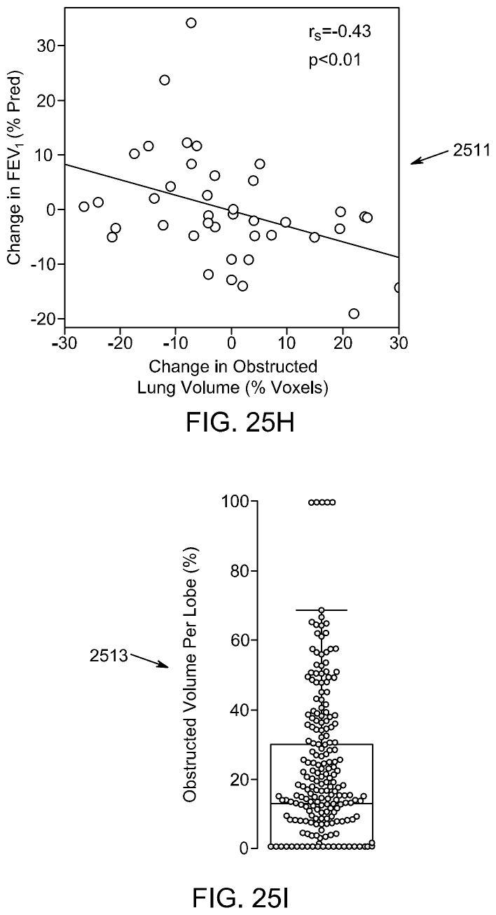

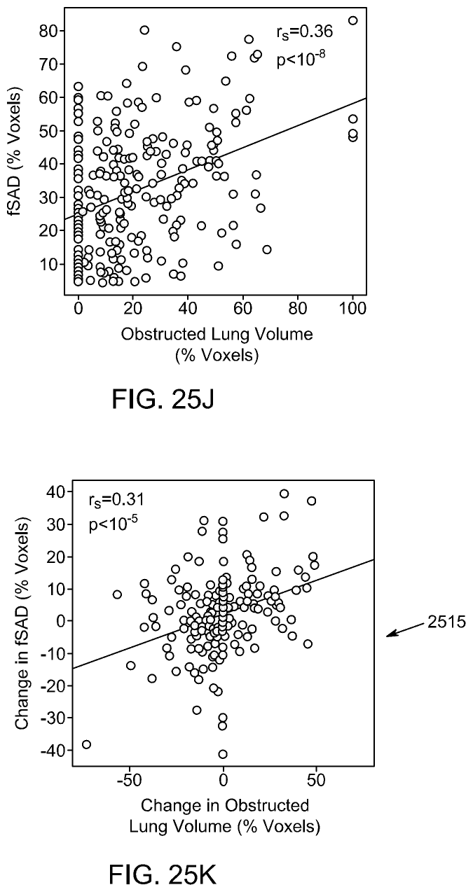

FIGS. 25A-25K also provide illustrations of experimental data. As shown in FIG. 25A, mucus plugs are associated with an increase in modeled airway resistance and in measured air trapping in lung regions distal to mucus-occluded airways. A schematic illustrating computation of resistance score (RS) by incorporating mucus plugging into the airway tree is shown. The airway tree 2501a is divided into different segments with an effective resistance Rn given by the length and radius of the airway at that location. After combining all segments, the net resistance of the airway tree in the presence of plugging (Rp) is compared with the resistance of the native airway tree in the absence of plugs (Rα) to yield the increased percentage in airway resistance RS=(100×[Rp−Rα]/Ra) due to plugs 2501b. An estimation of obstructed lung volume percentage (OLVP) is graphically represented 2503. The voxel volume of the lung region distal to a mucus occluded airway (Vo) was divided by the total voxel volume in the lobe (Vt) to generate the estimated obstructed lung volume percentage (100×Vo/Vt). Also shown is the distribution of RS for patients at baseline (n=54) 2505. The relationship between predicted RS and FEV1 at baseline and the relationship between changes in predicted RS and changes in FEV1 over 3 years for matched patients (n=40) 2507. Also shown is the distribution of OLVP per patient at baseline (n=53) 2509. The relationship between OLVP and FEV1 at baseline and the relationship between changes in predicted OLVP and changes in FEV1 over 3 years for matched patients (n=40) is provided 2511. Sensitivity analysis of outlier point (ΔRS=201, ΔFEV1=−14%) shows similar correlation coefficient (rs=−0.50, P=0.001 with outlier included and rs=−0.46, P=0.003 with outlier excluded). The distribution of OLVP per lobe at baseline (n=260) is plotted 2513. Also shown is the relationship between OLVP and disease probability measure functional small airways disease (DPM-fSAD) per lobe at baseline and the relationship between changes in OLVP and DPM-fSAD per lobe over 3 years (n=195) 2515, where rs denotes Spearman correlation coefficient.

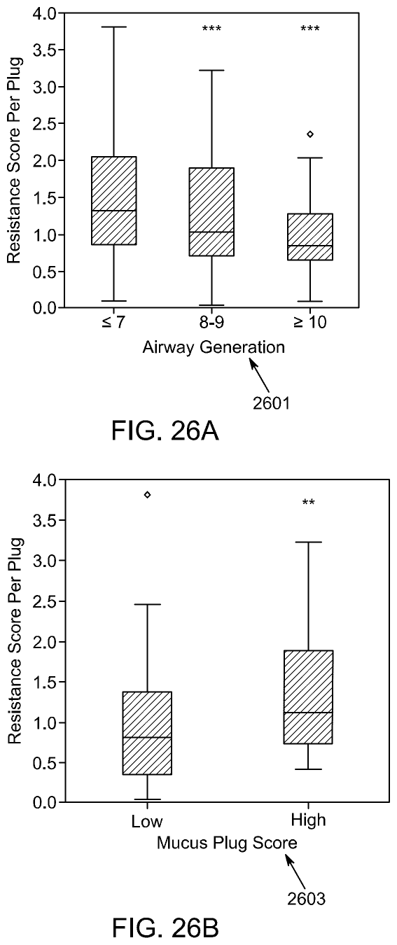

FIGS. 26A-26B illustrate experimental data showing that mucus plugs in proximal generations have a great effect on resistance score than distal generations. Depicted are resistance score (RS) per plug grouped independently by proximal airway generations (7 or less), intermediate airway generations (8 and 9), and distal airway generations (10 and greater) (n=1,327 plugs) 2601. The RS values for each generation group are shown for proximal, intermediate, and distal airway generations. ***P<0.001 by Kruskal-Wallis test; P=0.008 and P=0.002 for comparison with proximal versus intermediate and proximal versus distal generations, respectively, using linear mixed model with random effects for patient. Also shown is RS per plug grouped independently by mucus plug score-high (>11 plugs) and plug score-low (≤11 plugs) (n=94 patients). **P<0.01 by Kruskal-Wallis test; P=0.014 for linear mixed model with random effects for patient 2603.

Experimental data was used to generate a quantitative assessment of airway mucus plug pathology. The analysis of mucus plugs in CT lung scans in asthma presented in accordance with the systems and methods described herein yields multiple potentially novel quantitative measures of mucus plug pathology in the lung. These measures may serve as biomarkers of mucus pathology and can be quantitatively assessed in accordance with Quantitative Assessment of Airway Mucus Plug Pathology (qAAMP). All of the qAAMP measures can be generated in CT lung scans using the systems and methods described herein.

### Conclusion

Persistent mucus plugs in proximal airway generations occur in asthma and demonstrate a stochastic process of formation and resolution over time. Proximal airway mucus plugs are consequential for airflow and are in locations amenable to treatment by inhaled mucoactive drugs or bronchoscopy. Accordingly, data generated regarding mucus plugs can be used to develop treatment plans for mucoactive drugs or bronchoscopy.

Detailed size and shape information on 1,397 mucus plugs in 57 patients with asthma was generated and the airway tree locations occluded by these plugs and their lung function consequences were identified. Radiographically visible mucus plugs in asthma were hetero-geneous in their size and shape, and determined to be located primarily in 2 to 4 mm airways, and persist for many years, often in the same airway. Modeling data also indicates that mucus plugs increase airway resistance and air trapping in lung regions distal to mucus-occluded airways and provide strong rationale to treat mucus plugs as a strategy to improve airflow in asthma.

The length distribution of mucus plugs in asthma was determined to be multimodal, and best fit modeling indicated that a plug length of 12 mm defines short (“stubby”) and long (“stringy”) plug phenotypes. Although only 40% of the mucus plugs were stringy, these plugs contributed the most mucus volume in patients with the highest mucus burden. The heterogeneity of the number and size of mucus plugs has great relevance for the design of clinical trials that test interventions to treat mucus plugs. For example, it is likely that more numerous mucus plugs or plugs with a stringy phenotype will take longer to respond to treatment (especially inhaled treatments) than less numerous or stubby plugs. In addition, 3-year longitudinal data sets as generated herein can be used in the determination of the required duration of mucus plug treatments.

Generated data indicated that the same airway location has persistent plugging for 3 years and other cases where mucus plugs disappear from an airway over time or form in a new airway location. The average plug length and volume in these airways is centered around zero and have a normally distributed change in length and volume, indicating that these observed plugs persist in the airways and undergo a stochastic process of formation and resolution. These observations indicate that many patients with asthma have a persistent mucus plug phenotype that results from a dynamic balance of mucus plug persistence, resolution, and new formation. The generated data provides insight into the kinetic processes of airway mucus plugs and suggests that, while one-time removal of mucus plugs may have clinical benefit, repeated treatments may be needed to prevent formation of newly formed plugs in susceptible airways.

The lung image-based approach discussed herein shows that mucus plugs in asthma occur in airways that are 2-4 mm in diameter, and these airways include the fourth-and fifth-generation airways that aerate the proximal portions of bronchopulmonary segments. This finding that mucus plugs in asthma occur in segmental and larger subsegmental airways is important because they are likely to have larger effects on lung function in these proximal airway locations. Indeed, compared with mucus plugs in more distal airway locations, experimental data indicates that mucus plugs in proximal airway locations are more consequential for spirometry-based measures of lung function and model-based estimates of airway resistance. Removal of these mucus plugs is, therefore, a rational strategy to improve lung function in asthma. In this context, modeling of airway resistance in accordance with the methods described herein, which computes by comparing the resistance of the airway tree in the presence and absence of mucus plugs, can be thought of as a “virtual plug extraction” or simulation. Our virtual plug extraction data support removal of mucus plugs as a strategy to improve lung function in asthma. In other words, the described systems and methods can be used as a simulation tool allowing for further analysis of treatments.

Development of muco-active drugs for lung disease has been slowed by lack of predictive and monitoring biomarkers and by limited information about mucus plug phenotypes to guide drug dosing and formulation. The quantitative qAAMP metrics provided herein provide improved ability to select patients with mucus plug-high phenotypes for clinical trials of muco-active drugs and to monitor the effects of treatment on mucus plugs in these patients. For example, the qAAMP measures will allow determination of whether a muco-active treatment affects total mucus plug burden and whether this occurs globally in the airway tree or is restricted to specific locations in the airway tree. The disclosed systems and methods can be used to guide drug dosing and drug formulation. For example, the mucus plug volume data can be used to calculate the delivered drug dose required to lyse mucus plugs. In addition, the airway mucus plug map data and 3D visualizations of the location of persistent plugs can guide optimization of the physiochemical properties of aerosols or mechanical interventions needed to reach mucus plugs in fourth-to tenth-generation airways.

Although the present disclosure may provide a sequence of steps, it is understood that in some embodiments, additional steps may be added, described steps may be omitted, and the like. Additionally, the described sequence of steps may be performed in any suitable order.

While illustrative embodiments have been described herein, the scope thereof includes any and all embodiments having equivalent elements, modifications, omissions, combinations (e.g., of aspects across various embodiments), adaptations and/or alterations as would be appreciated by those in the art based on the present disclosure. For example, the number and orientation of components shown in the exemplary systems may be modified.

Thus, the foregoing description has been presented for purposes of illustration. It is not exhaustive and is not limiting to the precise forms or embodiments disclosed. Modifications and adaptations will be apparent to those skilled in the art from consideration of the specification and practice of the disclosed embodiments.

We claim:

1. A computer-implemented method, the method comprising:
receiving a plurality of images of one or both lungs of a subject from a computed tomography scan;
identifying one or more mucus plug portions within the received plurality of images;
determining a whole mucus plug associated with each of the identified mucus plug portions by applying a continuity algorithm to the received plurality of images;
generating at least one whole mucus plug metric for each of the determined whole mucus plugs, wherein the whole mucus plug metric comprises at least one of a length, a diameter, a volume, or a surface area;
generating at least one whole mucus plug phenotype for each of the determined whole mucus plugs;
generating subject-level whole mucus plugs metrics comprising at least one of an average length, an average diameter, an average volume, or an average surface area for the determined whole mucus plugs;
generating a subject-level mucus characterization data set comprising at least one of a whole mucus plug score, a total mucus plug volume, and a mucus plug slice score;
determining a subject-level quantitative assessment of airway mucus plug pathology based on the subject-level mucus characterization data set; and
determining a subject-level mucus plug phenotype based on the plurality of generated whole mucus plug phenotypes.

2. The computer-implemented method of claim 1, wherein generating the at least one whole mucus plug phenotype further comprises:
applying a threshold value to at least one of a length, a diameter, a volume, or a surface area, or a combination thereof, in order to determine a categorization of each mucus plug.

3. The computer-implemented method of claim 1, wherein generating a subject-level mucus characterization data set comprises determining at least one of an average length, an average diameter, an average volume, or an average surface area for the determined whole mucus plugs.

4. The computer implemented method of claim 1, further comprising:
predicting or monitoring the efficacy of treatments based on the determined subject-level quantitative assessment of airway mucus path pathology, wherein the treatment comprises at least one of a muco-active drug, an anti-inflammatory drug, interventional bronchoscopy, a cough assist device, or a vibrating vest.

5. The computer-implemented method of claim 1, further comprising:
predicting or monitoring the efficacy of treatments based on the determined subject-level mucus plug phenotype, wherein the treatment comprises at least one of a muco-active drug, an anti-inflammatory drug, interventional bronchoscopy, a cough assist device, or a vibrating vest.

6. The computer-implemented method of claim 1, wherein the whole mucus plug score is indicative of the total number of whole mucus plugs across the plurality of images associated with the subject.

7. The computer-implemented method of claim 1, wherein the total mucus plug volume comprises an aggregation of volume for each of the determined whole mucus plus for the subject.

8. The computer-implemented method of claim 1, wherein the mucus plug slice score comprises the sum of the mucus plug annotations across the received plurality of images.

9. The computer implemented method of claim 1, wherein the plug-level phenotype comprises a stubby mucus pattern where a stubby mucus pattern indicates at least one of a length less than a predetermined threshold or a ratio of length-diameter less than a predetermined threshold.

10. The computer implemented method of claim 1, wherein the plug-level phenotype comprises a stringy mucus pattern, wherein a stringy mucus pattern indicates a length greater than a second predetermined threshold or a ratio of length-diameter greater than a second predetermined threshold.

11. The computer-implemented method of claim 1, wherein the subject-level phenotype comprises of one or more plugs classified within each phenotype.

12. The computer-implemented method of claim 1, further comprising:
generating an airway mucus plug map depicting a specific airway location for the determined whole mucus plugs, wherein generating the airway mucus map further comprises determining the position of the one or more whole mucus plugs, or the absence of said one or more mucus plugs in relation to an airway tree.

13. The computer-implemented method of claim 12, wherein generating the airway mucus map further comprises:
segmenting the plurality of images to identify a plurality of pixels depicting the lung and a plurality of airways included in the lungs,
determining, for each whole mucus plug, a distance between the whole mucus plug and an endpoint of each airway in the plurality of airways,
assigning, based at least on the distance between the whole mucus plug and the endpoint of each airway, each whole mucus plug to an airway having a nearest airway termination point, and
depicting the plurality of airways and the one or more mucus plugs assigned to the airway having the nearest airway termination point.

14. The computer-implemented method of claim 12, further comprising:
skeletonizing the plurality of airways to determine a best fit centerline for each airway of the plurality of airways; and
determining, based at least on the skeletonized plurality of airways, the location of the endpoint of each airway.

15. The computer-implemented method of claim 12, wherein generating the airway mucus plug map further comprises depicting a hierarchy of the plurality of airways.

16. The computer-implemented method of claim 12, wherein generating the airway mucus plug map further comprises depicting a lobar location and generation of each airway of the plurality of airways.

17. The computer-implemented method of claim 1, wherein identifying one or more mucus plug portions within the received plurality of images further comprises receiving an annotation identifying at least the portion of the one or more mucus plug portions.

18. The computer-implemented method of claim 17, further comprising:
receiving, from a client device, one or more user inputs placing a mark around at least the portion of the mucus plug portions in the one or more received images; and
determining, based at least on the one or more user inputs, the annotation identifying one or more mucus plug portions in the one or more images.

19. The computer-implemented method of claim 1, wherein identifying one or more mucus plug portions within the received plurality of images further comprises applying a trained machine learning algorithm on the received plurality of images.

20. The computer-implemented method of claim 19, wherein the machine learning algorithm comprises a convolutional neural network.

21. The computer-implemented method of claim 19, wherein the machine learning algorithm comprises a transformer model.

22. The method of claim 1, wherein determining a whole mucus plug associated with each of the identified mucus plug portions comprises:
applying a set of algorithms to identify voxel-members of the whole mucus plug that form a region of interest, wherein the set of algorithms comprises one or more of a trained clustering algorithm, and sequential slicing annotation algorithms;
selecting one or more contiguous regions of interest.

23. The method of claim 1, wherein determining a whole mucus plug associated with each of the identified mucus plug portions comprises determining a region of interest for the identified mucus plug portion for each of image among the plurality of images, wherein the region of interest separates a foreground corresponding to the mucus plug portion and a background corresponding to a parenchyma of the lung based on cluster analysis.

24. The method of claim 1, wherein the plurality of images comprise any cross-sectional imaging modality computed tomography (CT) scan of the lung and wherein each image of the plurality of images comprises an axial, a coronal, or a sagittal slice of the computed tomography (CT) scan.

25. A computer-implemented method, the method comprising:
receiving a plurality of images of one or both lungs of a subject from a computed tomography scan;
identifying one or more mucus plug portions within the received plurality of images;
determining a whole mucus plug associated with each of the identified mucus plug portions by applying a continuity algorithm to the received plurality of images;
determining a specific airway location for the determined whole mucus plug; and
generating an airway mucus plug map depicting a specific airway location for the determined whole mucus plugs.

26. The computer-implemented method of claim 25, wherein generating the airway mucus map further comprises determining the position of the one or more whole mucus plugs, or the absence of said one or more whole mucus plugs in relation to an airway tree.

27. The computer-implemented method of claim 25, wherein generating the airway mucus map further comprises:
segmenting the plurality of images to identify a plurality of pixels depicting the lung and a plurality of airways included in the lungs,
determining, for each whole mucus plug, a distance between the whole mucus plug and an endpoint of each airway in the plurality of airways,
assigning, based at least on the distance between the whole mucus plug and the endpoint of each airway, each whole mucus plug to an airway having a nearest airway termination point; and
depicting the plurality of airways and the one or more mucus plugs assigned to the airway having the nearest airway termination point.

28. The computer-implemented method of claim 25, further comprising:
skeletonizing the plurality of airways to determine a best fit centerline for each airway of the plurality of airways; and
determining, based at least on the skeletonized plurality of airways, the location of the endpoint of each airway.

29. The computer-implemented method of claim 25, wherein generating the airway mucus plug map further comprises depicting a hierarchy of the plurality of airways.

30. The computer-implemented method of claim 25, wherein generating the airway mucus plug map further comprises depicting a lobar location and generation of each airway of the plurality of airways.
